(19) United States
(12) Patent Application Publication
(10) Pub. No.: US 2020/0399246 A1
(43) Pub. Date: Dec. 24, 2020

(54) CHARGE TRANSPORT MATERIAL, COMPOUND, AND ORGANIC LIGHT-EMITTING ELEMENT

(71) Applicants: KYUSHU UNIVERSITY, NATIONAL UNIVERSITY CORPORATION, Fukuoka-shi, Fukuoka (JP); KYULUX, INC., Fukuoka-shi, Fukuoka (JP)

(72) Inventors: Linsong CUI, Fukuoka-shi, Fukuoka (JP); Chihaya ADACHI, Fukuoka-shi, Fukuoka (JP); Keiro NASU, Fukuoka-shi, Fukuoka (JP); Ayataka ENDO, Fukuoka-shi, Fukuoka (JP); Shuo-Hsien CHENG, Fukuoka-shi, Fukuoka (JP); YuSeok YANG, Fukuoka-shi, Fukuoka (JP); Hiroko NOMURA, Fukuoka-shi, Fukuoka (JP)

(21) Appl. No.: 16/979,299

(22) PCT Filed: Mar. 13, 2019
(86) PCT No.: PCT/JP2019/010132

§ 371 Date: Sep. 9, 2020

(30) Foreign Application Priority Data
Mar. 13, 2018 (JP) 2018-045065

(51) Int. Cl. C07D 403/10; C07D 401/10; C07C 22/08; C07C 255/51; C07D 401/14; H01L 51/00

(52) U.S. Cl. CPC C07D 403/10; C07D 401/10; C07C 22/08; C07C 255/51; C07D 401/14; H01L 51/5072; H01L 51/0072; H01L 51/0074; H01L 51/0059; H01L 51/0069; H01L 51/0085; H01L 51/0067

(57) ABSTRACT

A charge transport material containing a compound represented by the following formula (1). R¹ and R² each are a fluoroalkyl group, Ar¹ and Ar² each are an aromatic ring, A¹ and A² each are an aryl group substituted with a group having a positive Hammett σp value or with a phenyl group, or are a substituted or unsubstituted heteroaryl group bonding to Ar¹ or Ar² via a carbon atom, n1 and n2 each are a natural number.

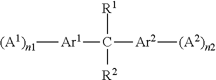

### TECHNICAL FIELD

The present invention relates to a compound useful as a charge transport material and to an organic light-emitting device using the compound.

### BACKGROUND ART

Studies for enhancing the light emission efficiency of organic light-emitting devices such as organic electroluminescent devices (organic EL devices) are being made actively. In particular, various kinds of efforts have been made for increasing light emission efficiency by newly developing and combining a light-emitting material, a host material, a hole transport material, an electro transport material and others to constitute an organic electroluminescent device. Among them, there is known a study relating to an organic electroluminescent device that utilizes a compound having a structure where two acceptor groups are bonded via a linking group.

For example, PTL 1 describes use of a compound represented by the following formula as an electron transport material for an organic electroluminescent device that emits blue phosphorescence.

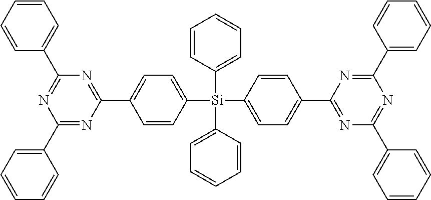 [SMILES: c1ccc(-c2nc(-c3ccccc3)nc(-c3ccc([Si](c4ccccc4)(c4ccccc4)c4ccc(-c5nc(-c6ccccc6)nc(-c6ccccc6)n5)cc4)cc3)n2)cc1]

PTL 2 describes use of a compound represented by the following formula as a material for an electron injection transport layer of an organic light-emitting device.

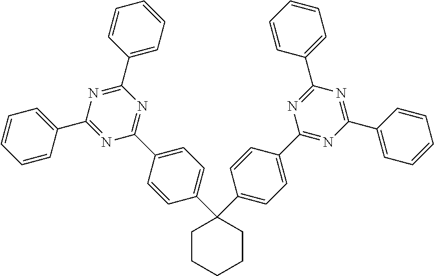 [SMILES: c1ccc(-c2nc(-c3ccccc3)nc(-c3ccc(C4(c5ccc(-c6nc(-c7ccccc7)nc(-c7ccccc7)n6)cc5)CCCCC4)cc3)n2)cc1]

In the compounds described in the above documents, the phenyl group substituted with a 4,6-diphenyl-1,3,5-triazin-2-yl group at both sides of the central diphenylsilylene group or cyclohexanediyl group corresponds to an acceptor group, and is considered to contribute toward electron transport by receiving an electron from other compounds.

### CITATION LIST

### Patent Literature

### PTL 1: Korean Patent 2012/0015138

### PTL 2: WO2016/068478

### SUMMARY OF INVENTION

### Technical Problem

As described above, PTLs 1 and 2 describe use of compounds having a structure where two phenyl groups each substituted with a 4,6-diphenyl-1,3,5-triazin-2-yl group are bonded via a linking group, as an electron transport material. However, the present inventors investigated these compounds in point of the performance thereof as a host material for a light-emitting layer, and have found that the compounds are insufficient as a host material.

Accordingly, the present inventors have taken particular note of a group of compounds having two acceptor groups especially in point of the structure of the linking group therein, and have exhaustively investigated the performance of the compounds as a host material, and as a result, have found that, in the case where the two acceptor groups are bonded via a methylene group substituted with an alkyl group, the kind of the substituent for the alkyl group has a significant influence on the performance of the compounds as a host material. Regarding this point, PTLs 1 and 2 do not make any detailed investigations relating to the kind of the substituent for the alkyl group in the case where the linking group is substituted with an alkyl group. Consequently, from these documents, no one can anticipate as to whether or not the performance of a compound, in which two acceptors groups are bonded via a methylene group, as a host material would be influenced by the kind of the substituent for the alkyl group that substitutes for the methylene group.

Given the situation, the present inventors have further made assiduous studies for the purpose of deriving a formula of a compound which has a structure where two acceptor groups are bonded via a linking group and which exhibits an excellent performance as a charge transport material such as a host material, and generalizing the structure of an excellent organic light-emitting device.

### Solution to Problem

As a result of assiduous studies, the present inventors have found that, when a methylene group substituted with a fluoroalkyl group is employed as a linking group that links two acceptor groups, an excellent performance as a charge transport material can be achieved. With that, the present inventors have reached a finding that, when such a compound is used as a charge transport material, an excellent organic light-emitting device can be provided. The present invention has been proposed on the basis of this finding, and specifically has the following constitution.

[1] A charge transport material containing a compound represented by the following formula (1):

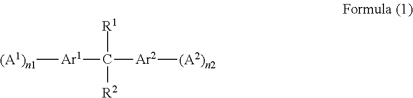

In the formula (1), R¹ and R² each independently represent a fluoroalkyl group, Ar¹ and Ar² each independently represent an aromatic ring optionally having a substituent, A¹ and A² each independently represent an aryl group substituted with a group having a positive Hammett's σp value, or an aryl group substituted with a phenyl group, or represent a substituted or unsubstituted heteroaryl group bonding to Ar¹ or Ar² via a carbon atom, n1 represents a natural number not more than the maximum number of substituents substitutable on Ar¹, n2 represents a natural number not more than the maximum number of substituents substitutable on Ar².

[2] The charge transport material according to [1], wherein R¹ and R² each are independently a perfluoroalkyl group.
[3] The charge transport material according to [1] or [2], wherein the carbon number of R¹ and R² is each independently 1 to 3.
[4] The charge transport material according to [3], wherein the carbon number of R¹ and R² is each independently 1 or 2.
[5] The charge transport material according to [3], wherein the carbon number of R¹ and R² is 1.
[6] The charge transport material according to [1], wherein R¹ and R² each are a trifluoromethyl group.
[7] The charge transport material according to any one of [1] to [6], wherein Ar¹ and Ar² each are independently a benzene ring optionally having a substituent.
[8] The charge transport material according to any one of [1] to [6], wherein Ar¹ and Ar² each are a benzene ring unsubstituted at the bonding positions other than the bonding positions to A¹ or A² and the bonding position to C to which R¹ and R² bond.
[9] The charge transport material according to any one of [1] to [8], wherein A¹ and A² each are independently a substituted or unsubstituted heteroaryl group bonding to Ar¹ or Ar² via a carbon atom.
[10] The charge transport material according to [9], wherein the substituted or unsubstituted heteroaryl group is a group containing at least one or more of a pyridine ring, a pyrimidine ring and a triazine ring.
[11] The charge transport material according to [9], wherein the substituted or unsubstituted heteroaryl group is a substituted or unsubstituted pyridinyl group, a substituted or unsubstituted pyrimidinyl group, or a substituted or unsubstituted triazinyl group.
[12] The charge transport material according to [9], wherein the substituted or unsubstituted heteroaryl group is a group containing a triazine ring.
[13] The charge transport material according to [9], wherein the substituted or unsubstituted heteroaryl group is a substituted or unsubstituted triazinyl group.
[14] The charge transport material according to [9], wherein the heteroaryl group is a heteroaryl group substituted with a substituted or unsubstituted aryl group.
[15] The charge transport material according to [9], wherein the heteroaryl group is a triazinyl group substituted with a substituted or unsubstituted aryl group.
[16] The charge transport material according to any one of [1] to [15], wherein A¹ and A² are the same groups.
[17] The charge transport material according to any one of [1] to [16], wherein n1 and n2 each are 1 or 2.
[18] The charge transport material according to any one of [1] to [17], wherein the charge transport material is a host material.
[19] The charge transport material according to any one of [1] to [17], wherein the charge transport material is an electron transport material.
[20] The charge transport material according to any one of [1] to [19], having a lowest excited triplet energy level (E_{T1}) of 2.90 eV or more.
[21] The charge transport material according to any one of [1] to [20], which is for an organic light-emitting device having a maximum emission wavelength of 360 to 495 nm.
[22] A compound represented by the following formula (1):

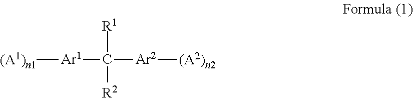

In the formula (1), R¹ and R² each independently represent a fluoroalkyl group, Ar¹ and Ar² each independently represent an aromatic ring optionally having a substituent, A¹ and A² each independently represent an aryl group substituted with a group having a positive Hammett's σp value, or an aryl group substituted with a phenyl group, or represent a substituted or unsubstituted heteroaryl group bonding to Ar¹ or Ar² via a carbon atom (but excluding a substituted or unsubstituted imidazolyl group bonding to Ar¹ or Ar² via a carbon atom, a substituted or unsubstituted thiadiazolyl group bonding to Ar¹ or Ar² via a carbon atom, and a substituted or unsubstituted oxadiazolyl group bonding to Ar¹ or Ar² via a carbon atom), n1 represents a natural number not more than the maximum number of substituents substitutable on Ar¹, n2 represents a natural number not more than the maximum number of substituents substitutable on Ar².

[23] An organic light-emitting device having a layer containing a compound represented by the following formula (1), on a substrate:

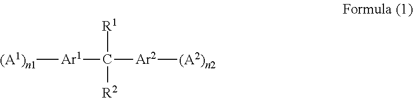

In the formula (1), R¹ and R² each independently represent a fluoroalkyl group, Ar¹ and Ar² each independently represent an aromatic ring optionally having a substituent, A¹ and A² each independently represent an aryl group substituted with a group having a positive Hammett's σp value, or an aryl group substituted with a phenyl group, or represent a substituted or unsubstituted heteroaryl group bonding to Ar¹ or Ar² via a carbon atom (but excluding a substituted or unsubstituted imidazolyl group bonding to Ar¹ or Ar² via a carbon atom, a substituted or unsubstituted thiadiazolyl group bonding to Ar¹ or Ar² via a carbon atom, and a substituted or unsubstituted oxadiazolyl group bonding to Ar¹ or Ar² via a carbon atom), n1 represents a natural number not more than the maximum number of substituents substitutable on Ar¹, n2 represents a natural number not more than the maximum number of substituents substitutable on Ar².

[24] The organic light-emitting device according to [23], containing a compound such that a difference ΔE_{ST} between the lowest excited singlet energy level and the lowest excited triplet energy level thereof is 0.3 eV or less, in the light-emitting layer.
[25] The organic light-emitting device according to [23] or [24], which emits delayed fluorescence.
[26] The organic light-emitting device according to any one of [23] to [25], having the compound represented by the formula (1) in the light-emitting layer.
[27] The organic light-emitting device according to [26], wherein the content of the compound represented by the formula (1) in the light-emitting layer is 50% by weight or more.
[28] The organic light-emitting device according to any one of [23] to [25], having the compound represented by the formula (1) in a layer formed between the light-emitting layer and the cathode.

### Advantageous Effects of Invention

The compound of the present invention is useful as a charge transport material. The organic light-emitting device using the compound of the present invention as a charge transport material can realize at least one of a low drive voltage, a high light emission efficiency and a long lifetime.

### BRIEF DESCRIPTION OF DRAWINGS

FIG. 1 This is a schematic cross-sectional view showing a layer configuration example of an organic electroluminescent device.

FIG. 2 This is a UV-visible ray absorption spectrum, a photoluminescence spectrum and a phosphorescence spectrum of a toluene solution of a compound 1.

FIG. 3 This is a graph showing an external quantum efficiency (EQE)-current density characteristic of an organic electroluminescent device using a compound 1.

FIG. 4 This is a graph showing a time-dependent change of a luminance ratio L/L₀ of an organic electroluminescent device using a compound 1.

### DESCRIPTION OF EMBODIMENTS

The contents of the invention will be described in detail below. The constitutional elements may be described below with reference to representative embodiments and specific examples of the invention, but the invention is not limited to the embodiments and the examples. In the description herein, a numerical range expressed as “to” means a range that includes numerical values before and after “to” as the lower limit and the upper limit. In the invention, the hydrogen atom that is present in the molecule of the compound used in the invention is not particularly limited in isotope species, and for example, all the hydrogen atoms in the molecule may be ¹H, and all or a part of them may be ²H (deuterium D).

### [Compound Represented by Formula (1)]

The charge transport material of the present invention is characterized by containing a compound represented by the following formula (1):

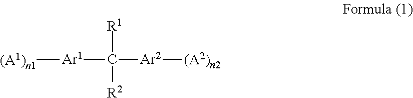

In the formula (1), R¹ and R² each independently represent a fluoroalkyl group. “Fluoroalkyl group” in the present invention is a group having a structure of an alkyl group, at least one hydrogen atom of which is substituted with a fluorine atom. The fluoroalkyl group represented by R¹ and R² may be a perfluoroalkyl group in which all hydrogen atoms of the alkyl group are substituted with fluorine atoms, or may be a partially fluorinated alkyl group in which only a prat of hydrogen atoms of the alkyl group are substituted with fluorine atoms. Among these, the fluoroalkyl group is preferably a perfluoroalkyl group, and the carbon number of the fluoroalkyl group is preferably any of 1 to 20, more preferably any of 1 to 10, even more preferably any of 1 to 5, further more preferably any of 1 to 3, still further more preferably 1 or 2, and especially more preferably 1. Most preferably, the fluoroalkyl group represented by R¹ and R² is a trifluoromethyl group. When the carbon number of the fluoroalkyl group is 3 or more, the fluoroalkyl group may be linear or branched. The fluoroalkyl groups represented by R¹ and R² may be the same as or different from each other. Examples of the case where the fluoroalkyl groups represented by R¹ and R² are different from each other include a case where the number of carbon atoms and fluorine atoms differs, a case differing in point of linear or branched, and a case differing in the number of branches or in the branching position of a branched fluoroalkyl group.

Ar¹ and Ar² each independently represent an aromatic ring optionally having a substituent. “Aromatic ring” constituting Ar¹ and Ar² is an aromatic ring not containing a hetero atom, and is a cyclic structure derived from an aromatic hydrocarbon by removing the hydrogen atom at a position corresponding to the bonding position to the other group. Ar¹ and Ar² may be the same or different, but are preferably the same. The aromatic ring for Ar¹ and Ar² may be a single ring or may be a condensed ring formed by condensation of 2 or more aromatic rings. The carbon number of the aromatic ring is preferably 6 to 22, more preferably 6 to 18, even more preferably 6 to 14, further more preferably 6 to 10. Specific examples of the aromatic ring include a benzene ring, a naphthalene ring, and an anthracene ring, and a benzene ring is preferred. In the aromatic ring, the position except the bonding position to A¹ or A² and the bonding position to C to which R¹ and R² are bonded may be substituted with a substituent or may be unsubstituted, and is preferably unsubstituted. Specifically, it is most preferable that Ar¹ and Ar² each are a benzene ring unsubstituted at the position except the the bonding position to A¹ or A² and the bonding position to C to which R¹ and R² are bonded.

In the aromatic ring of Ar¹ and Ar², examples of the substituent that may substitute at the position except the bonding position to A¹ or A² and the bonding position to C to which R¹ and R² are bonded include a hydroxy group, a halogen atom, an alkyl group having 1 to 20 carbon atoms, an alkoxy group having 1 to 20 carbon atoms, an alkylthio group having 1 to 20 carbon atoms, an alkyl-substituted amino group having 1 to 20 carbon atoms, an aryl-substituted amino group having 1 to 20 carbon atoms, an aryl group having 6 to 40 carbon atoms, a heteroaryl group having 3 to carbon atoms, an alkenyl group having 2 to 10 carbon atoms, an alkynyl group having 2 to 10 carbon atoms, an alkylamide group having 2 to 20 carbon atoms, an arylamide group having 7 to 21 carbon atoms, and a trialkylsilyl group having 3 to 20 carbon atoms. Among these specific examples, those further substitutable with any other substituent may be substituted with such a substituent. More preferred substituents include a alkyl group having 1 to 20 carbon atoms, an alkoxy group having 1 to 20 carbon atoms, an alkylthio group having 1 to 20 carbon atoms, an alkyl-substituted amino group having 1 to 20 carbon atoms, an aryl-substituted amino group having 1 to 20 carbon atoms, an aryl group having 6 to 40 carbon atoms, and a heteroaryl group having 3 to 40 carbon atoms.

A¹ and A² each independently represent an aryl group substituted with a group having a positive Hammett's σp value, or an aryl group substituted with a phenyl group, or represent a substituted or unsubstituted heteroaryl group bonding to Ar¹ or Ar² via a carbon atom.

Here, “Hammett's σₚ value” is one propounded by L. P. Hammett's, and is one to quantify the influence of a substituent on the reaction rate or the equilibrium of a para-substituted benzene derivative. Specifically, the value is a constant (σₚ) peculiar to the substituent in the following equation that is established between a substituent and a reaction rate constant or an equilibrium constant in a para-substituted benzene derivative:

log(k/k₀)=ρσₚ

or

log(K/K₀)=ρσₚ

In the above equations, k represents a rate constant of a benzene derivative not having a substituent; k₀ represents a rate constant of a benzene derivative substituted with a substituent; K represents an equilibrium constant of a benzene derivative not having a substituent; K₀ represents an equilibrium constant of a benzene derivative substituted with a substituent; ρ represents a reaction constant to be determined by the kind and the condition of reaction. Regarding the description relating to the “Hammett's σₚ value” and the numerical value of each substituent, reference may be made to the description relating to σₚ value in Hansch, C., et. al., Chem. Rev., 91, 165-195 (1991). A substituent having a negative Hammett's σₚ value tends to exhibit electron-donating performance (donor-like performance) and a substituent having a positive Hammett's σₚ value tends to exhibit electron-accepting performance (acceptor-like performance). In the following description, “having a negative Hammett's σₚ value” may be expressed as “electron-donating”, and “having a positive Hammett's σₚ value” may be expressed as “electron-accepting”.

n1 represents a number of A¹'s substitutable on the aromatic ring that constitutes Ar¹, and is a natural number not more than the maximum number of substituents substitutable on Ar¹. n2 represents a number of A²'s substitutable on the aromatic ring that constitutes Ar², and is a natural number not more than the maximum number of substituents substitutable on Ar². A¹ and A² may be the same or different, but are preferably the same. When n1 is 2 or more, plural A¹'s may be the same or different, but are preferably the same. When n2 is 2 or more, plural A²'s may be the same or different, but are preferably the same.

Regarding the aryl group substituted with a group having a positive Hammett σp value, and the aryl group substituted with a phenyl group that are represented by A¹ and A², the aromatic ring to constitute the aryl group may be a single ring, or may also be a condensed ring formed through condensation of 2 or more aromatic rings, or a linked ring formed by linking 2 or more aromatic rings. In the case where 2 or more aromatic rings are linked, they may be linked linearly or in a branched manner. The carbon number of the aromatic ring to constitute the aryl group is preferably 6 to 22, more preferably 6 to 18, even more preferably 6 to 14, further more preferably 6 to 10. Specific examples of the aryl group include a phenyl group, a naphthyl group and a biphenyl group, and a phenyl group is preferred.

Regarding the aryl group substituted with a group having a positive Hammett σp value, the number of the group having a positive Hammett σp value that substitutes on the aryl group may be 1 or may be 2 or more, but is preferably 1 to 3, more preferably 1 or 2. When the substitution number of the groups having a positive Hammett σp value on the aryl group is 2 or more, the plural groups having a positive Hammett σp value may be the same as or different from each other, but are preferably the same.

Specific examples of the group having a positive Hammett σp value that substitutes on the aryl group include a cyano group, a nitro group, a halogen atom, a formyl group, a carbonyl group, an alkoxycarbonyl group, a haloalkyl group, and a sulfonyl group, and a cyano group is preferred. In addition, a substituted or unsubstituted heteroaryl group that bonds to Ar¹ or Ar² via a carbon atom to be mentioned hereinunder, and specific examples to be expressed by the formulae shown hereinunder are also preferably usable as the group having a positive Hammett σp value.

Regarding the aryl group substituted with a phenyl group, the number of the phenyl groups substitutable on the aryl group may be one or may be 2 or more, but is preferably 1 to 3, more preferably 1 or 2.

The substituted or unsubstituted heteroaryl group bonding to Ar¹ or Ar² via a carbon atoms, as represented by A¹ and A², is preferably a group having a positive Hammett σp value, and the aromatic hetero ring that the heteroaryl group includes is preferably a π-electron-deficient aromatic hetero ring.

In the substituted or unsubstituted heteroaryl group bonding to Ar¹ or Ar² via a carbon atoms, as represented by A¹ and A², the hetero atom that the heteroaryl group has includes a nitrogen atom, an oxygen atom, a sulfur atom, and a boron atom, and the heteroaryl group preferably contains at least one nitrogen atom as a ring member. Such a heteroaryl group includes a 5-membered or 6-membered ring having a nitrogen atom as a ring member, and a group having a structure where a benzene ring is condensed with a 5-membered or 6-membered ring having a nitrogen atom as a ring member. A group containing any one or more of a pyridine ring, a pyrazine ring, a pyrimidine ring, a pyridazine ring and a triazine ring is preferred, a group containing any one or more of a pyridine ring, a pyrimidine ring, and a triazine ring is more preferred, and a group containing a triazine ring is even more preferred. Specific examples of the heteroaryl group include a monovalent group formed by removing one hydrogen atom from a pyridine ring, a pyrazine ring, a pyrimidine ring, a pyridazine ring or a triazine ring, a group having a condensed structure of any of those aromatic hetero rings, and a group having a structure where a benzene ring is condensed with any of those aromatic hetero rings; and a substituted or unsubstituted pyridinyl group, a substituted or unsubstituted pyrimidinyl group, or a substituted or unsubstituted triazinyl group is preferred, and a substituted or unsubstituted triazinyl group is more preferred. The heteroaryl group bonding to Ar¹ or Ar² via a carbon atom may be substituted with a substituent or may be unsubstituted, but is preferably substituted with a substituent. The number of the substituents on the heteroaryl group may be 1 or may be 2 or more, but is preferably 1 to 3, more preferably 1 or 2. When the heteroaryl group has 2 or more substituents, the plural substituents may be the same as or different from each other, but are preferably the same.

Examples of the substituent substitutable on the heteroaryl group that bonds to Ar¹ or Ar² via a carbon atom includes an alkyl group, an aryl group, a cyano group, a halogen atom and a heteroaryl group, and among these, the alkyl group, the aryl group and the heteroaryl group each are preferably an alkyl group having 1 to 20 carbon atoms, an aryl group having 6 to 40 carbon atoms, and a heteroaryl group having 5 to 40 carbon atoms, respectively. Among these, a preferred substituent for the heteroaryl group is an aryl group. The aromatic ring to constitute the aryl group may be a single ring or may be a condensed ring formed through condensation of 2 or more aromatic rings, or may also be a linked ring formed by linking 2 or more aromatic rings. In the case where 2 or more aromatic rings are linked, they may be linked linearly or in a branched manner. The carbon number of the aromatic ring to constitute the aryl group is preferably 6 to 22, more preferably 6 to 18, even more preferably 6 to 14, further more preferably 6 to 10. Specific examples of the aryl group include a phenyl group, a naphthyl group and a biphenyl group, and a phenyl group is most preferred. Among these substituents, those substitutable with a substituent may be substituted with any of these substituents.

In A¹ and A², the substituted or unsubstituted heteroaryl group bonding to Ar¹ or Ar² via a carbon atom is preferably a group represented by the following formula (2):

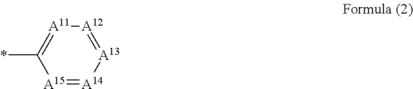

In the formula (2), A¹¹ to A¹⁵ each independently represent N or C(R¹⁹), R¹⁹ represents a hydrogen atom or a substituent. At least one of A¹¹ to A¹⁵ is N, and preferably one to three thereof are N, most preferably three are N. Among A¹¹ to A¹⁵, preferably at least one of A¹¹, A¹³ and A¹⁵ is N, more preferably A¹¹, A¹³ and A¹⁵ are all N. Also preferably, at least one of A¹² and A¹⁴ is C(R¹⁹) and R⁹ is a substituent, more preferably A¹² and A¹⁴ are both C(R¹⁹) and R¹⁹ is a substituent. When the group represented by the formula (2) has plural R¹⁹'s, the plural R¹⁹'s may be the same as or different from each other, but are preferably the same. Regarding the preferred range and specific examples of the substituent that R¹⁹ may have, reference may be made to the preferred range and specific examples of the substituent that may be substitutable on the heteroaryl group bonding to Ar¹ or Ar² via a carbon atom mentioned hereinabove. * indicates a bonding position to Ar¹ or Ar² in the formula (1).

n1 represents a number of A¹'s substitutable on the aromatic ring that constitutes Ar¹, and is a natural number not more than the maximum number of substituents substitutable on Ar¹. n2 represents a number of A²'s substitutable on the aromatic ring that constitutes Ar², and is a natural number not more than the maximum number of substituents substitutable on Ar². The substitutable position on the aromatic ring is specifically a methine group (—CH═) to constitute the aromatic ring, and “the maximum number of substitutable substituents” as referred to herein corresponds to a number to be calculated by subtracting 1 from the number of the methine groups constituting the aromatic ring. For example, in the case where Ar¹ and Ar² each are a benzene ring, the maximum number of substitutable substituents is 5, and in this case, n1 and n2 each can be any number of 1 to 5, but each is preferably 1 to 3, more preferably 1 or 2, even more preferably 1. n1 and n2 may be the same or different, but are preferably the same. Here, in the case where Ar¹ and Ar² each are a benzene ring and n1 and n2 are 1, the benzene ring constitutes a phenylene group that links A¹ or A² to C to which R¹ and R² bond. The phenylene group that the benzene ring constitutes may be any of a 1,2-phenylene group, a 1,3-phenylene group or a 1,4-phenylene group, but is preferably a 1,4-phenylene group.

In the following, as represented by A¹ and A², specific examples (A-1 to A-77) of the group having a positive Hammett σp value in the “aryl group substituted with a group having a positive Hammett σp value”, and specific examples (those of A-1 to A-77 bonding to Ar¹ or Ar² via the carbon atom of the aromatic hetero ring) of the substituted or unsubstituted heteroaryl group bonding to Ar¹ or Ar² via a carbon atom are exemplified. However, in the present invention, the groups that A¹ and A² may represent should not be limitatively interpreted by these exemplifications. In the following formulae, * indicates a bonding position to the aryl group in the aryl group substituted with a group having a positive Hammett σp value. Further, * extending from the carbon atom of the aromatic hetero ring also indicates a bonding position to Ar¹ or Ar². In the case where the formula has plural *'s, one of the plural *'s is a bonding position to the aryl group, or a bonding position to Ar¹ or Ar². The other remaining *'s indicate a hydrogen atom or a substituent. Regarding the preferred range and specific examples of the substituent, reference may be made to the preferred range and specific examples of the substituent substitutable on the heteroaryl group bonding to Ar¹ or Ar² via a carbon atom described hereinabove, but * that A¹ includes is also preferably a substituent satisfying the requirement for (A²)ₙ₂-Ar²—C(R¹)(R²)— or a substituent satisfying the requirement for (A²)ₙ₂-Ar²— or a substituent satisfying the requirement for A² in the formula (1), and among these, more preferably a substituent satisfying the requirement for (A²)ₙ₂-Ar²—C(R¹)(R²)— in the formula (1). Also preferably, * that A² includes is a substituent satisfying the requirement for (A¹)ₙ₁-Ar¹—C(R¹)(R²)— or a substituent satisfying the requirement for (A¹)ₙ₁-Ar¹— or a substituent satisfying the requirement for A¹ in the formula (1), and among these, more preferably a substituent satisfying the requirement for (A¹)ₙ₁-Ar¹—C(R¹)(R²)— in the formula (1).

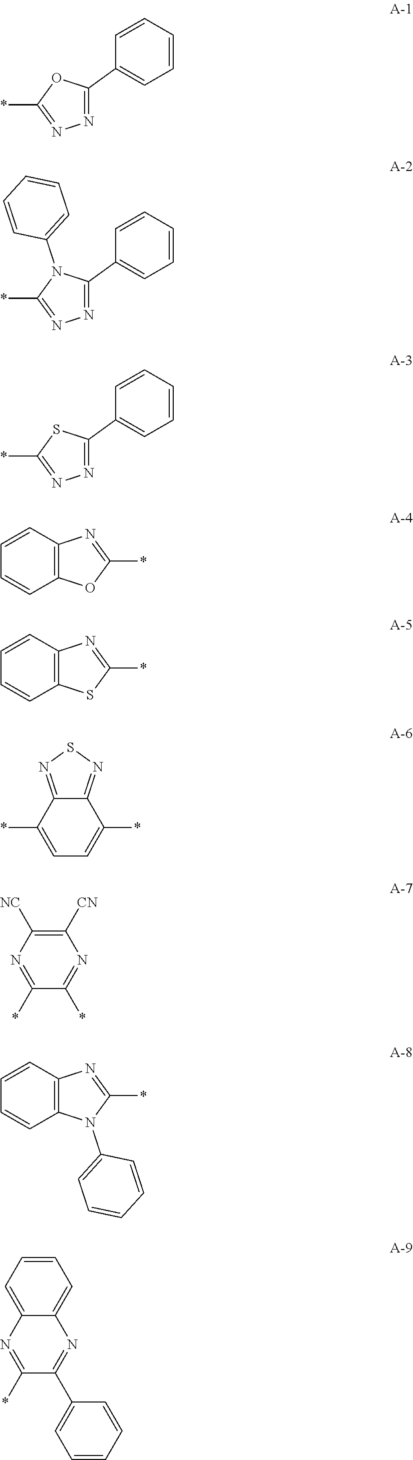

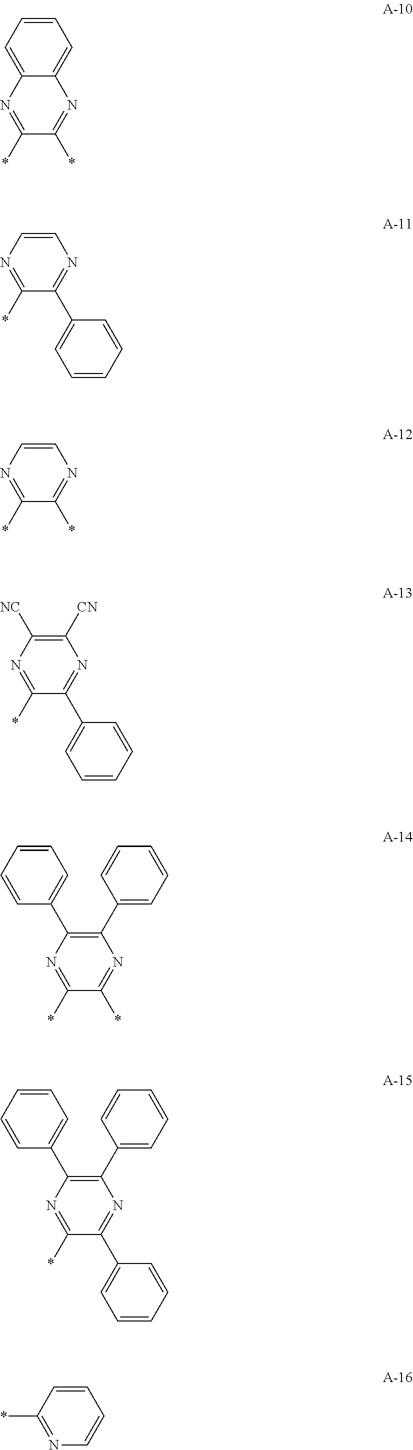

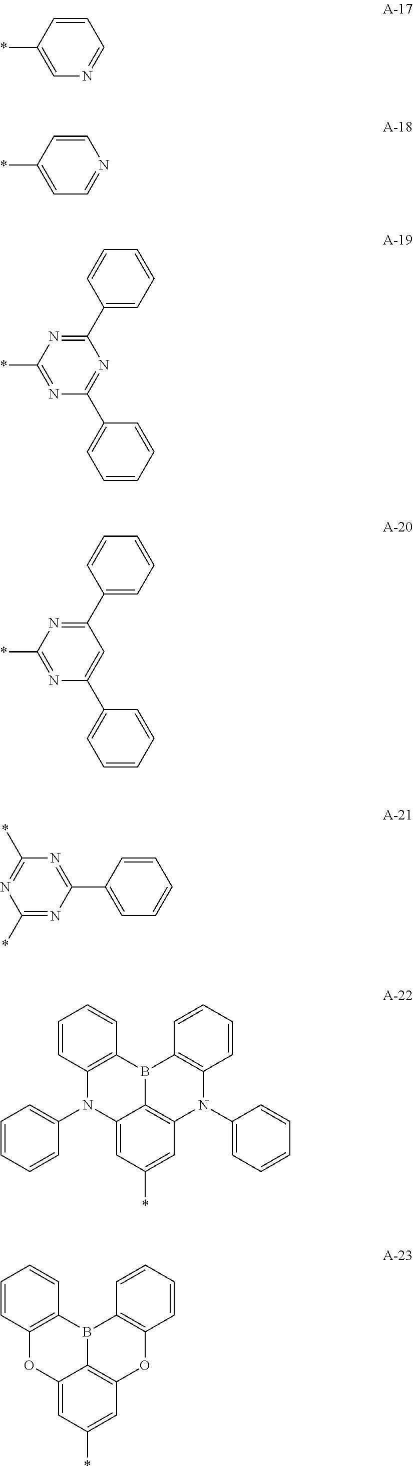

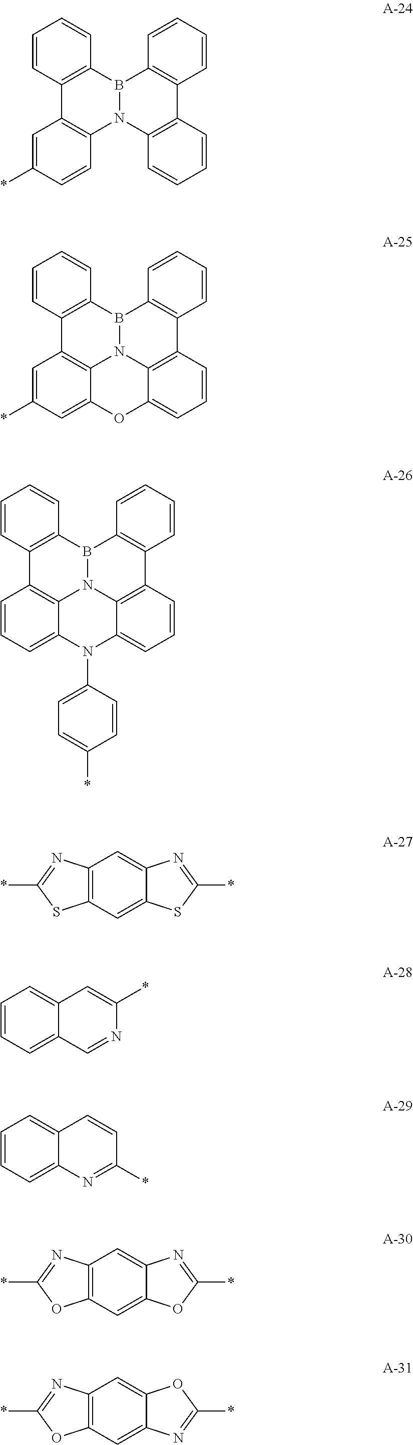

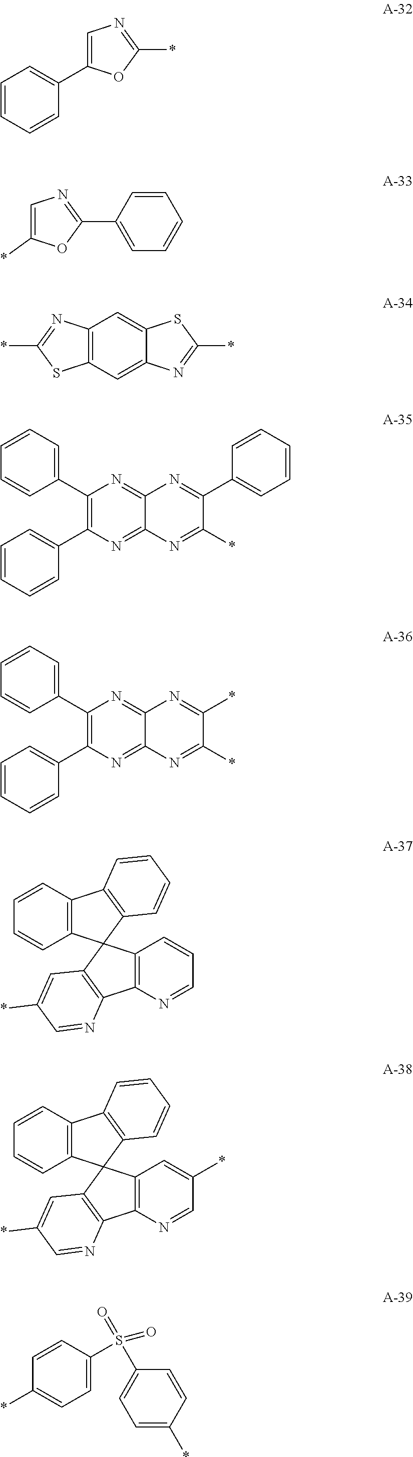

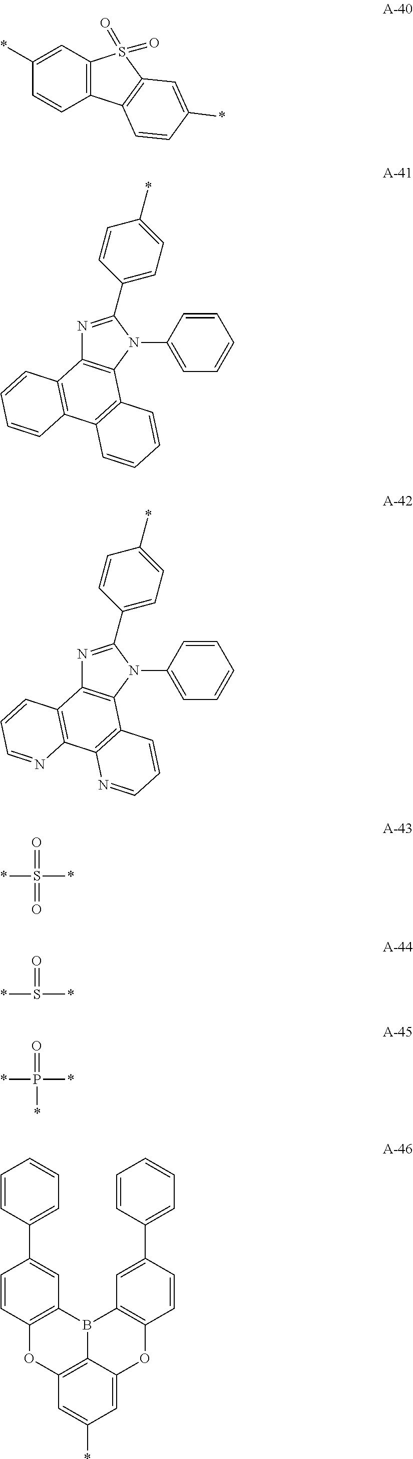

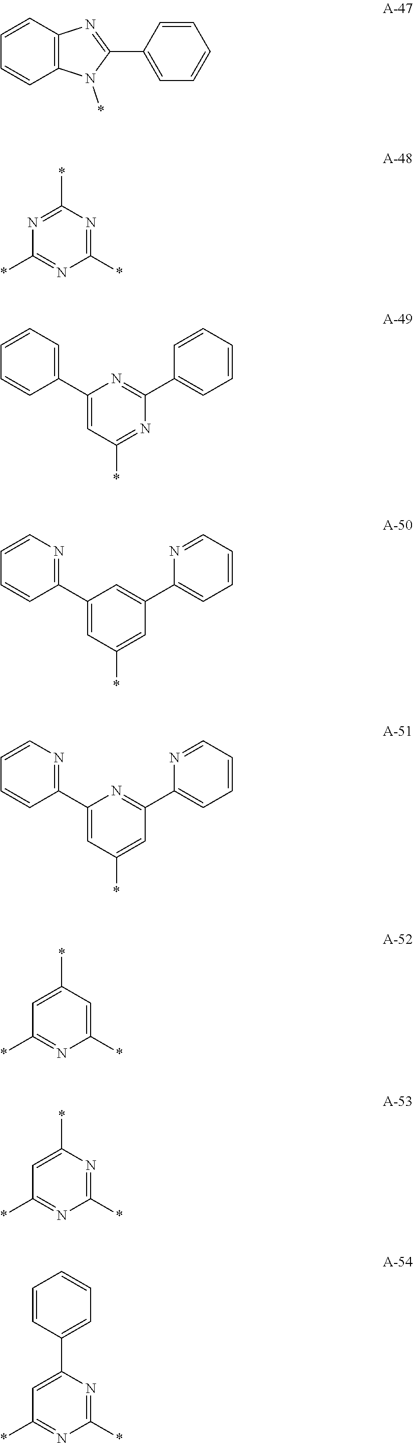

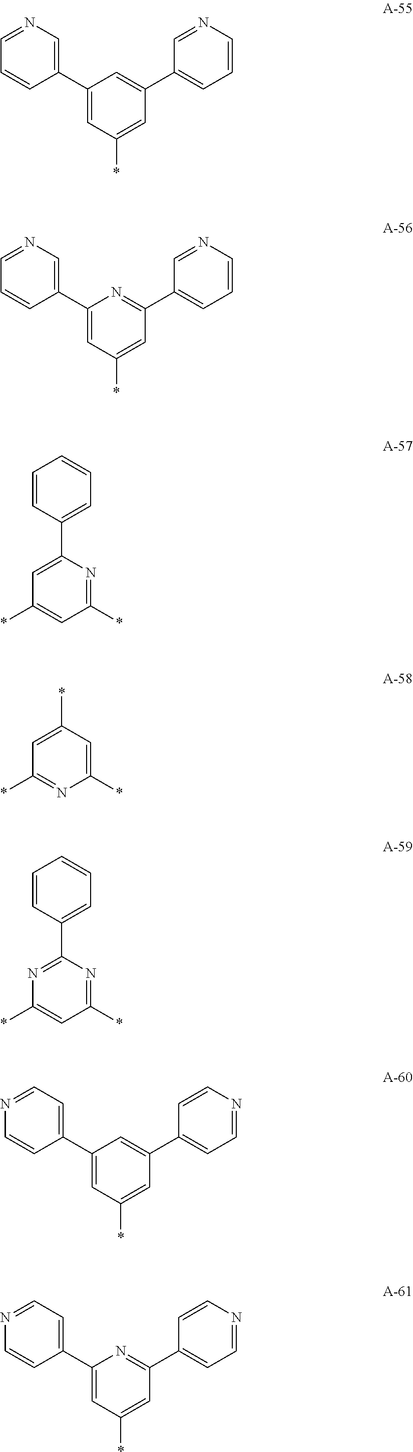

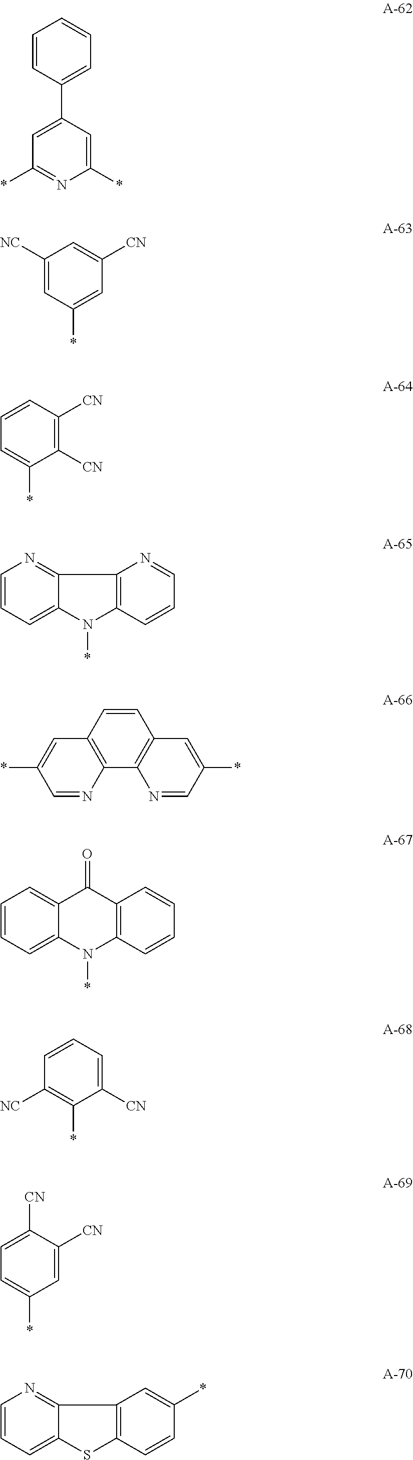

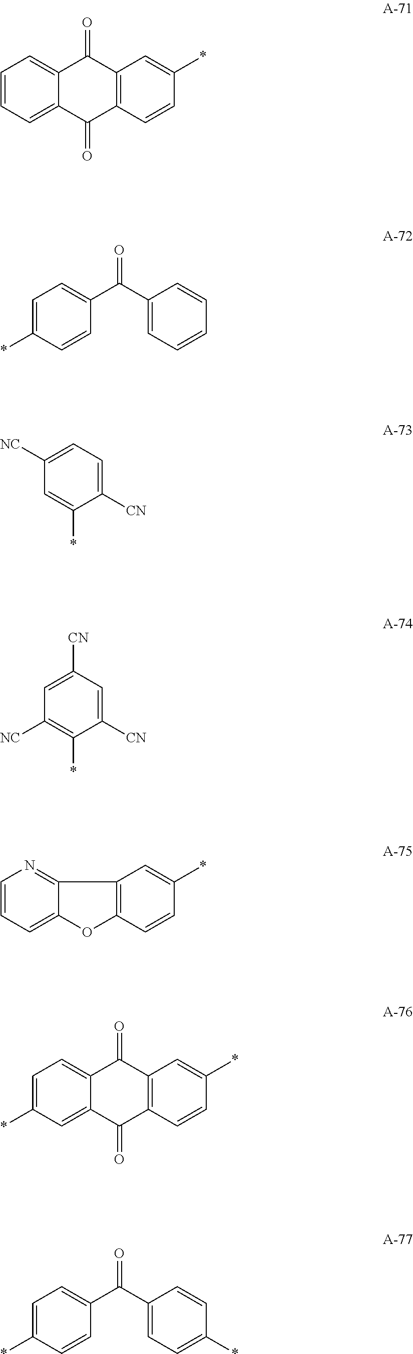

Preferably, (A¹)ₙ₁-Ar¹— and (A²)ₙ₂-Ar²— in the formula (1) each are an aryl group substituted with a heteroaryl group substituted with a substituted or unsubstituted aryl group, more preferably an aryl group substituted with a triazinyl group substitute with a substituted or unsubstituted aryl group, even more preferably a phenyl group substituted with a triazinyl group substitute with a substituted or unsubstituted phenyl group.

Specific examples of the compound represented by the formula (1) are shown below. However, the compound represented by the formula (1) usable in the present invention should not be limitatively interpreted by these specific examples.

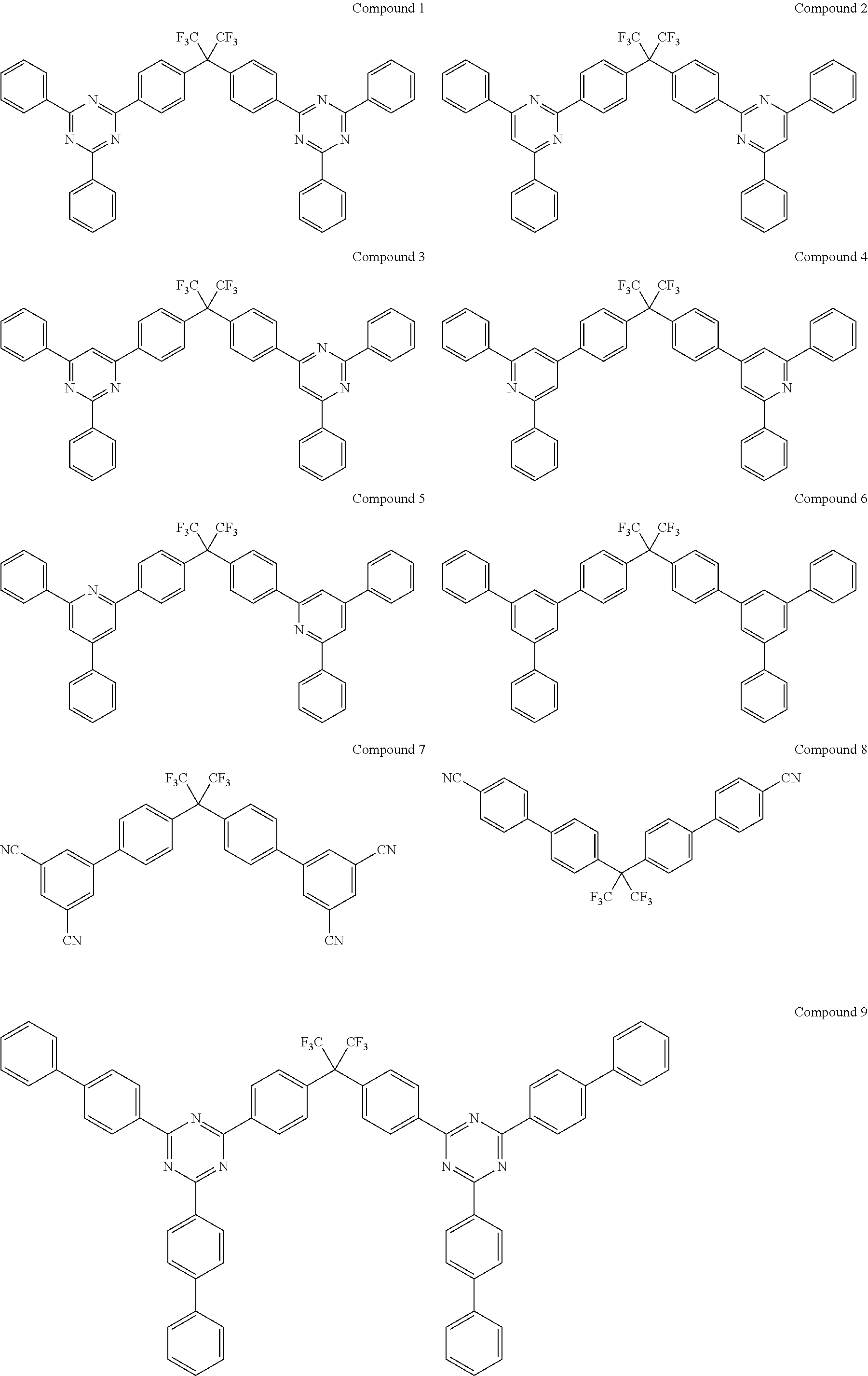

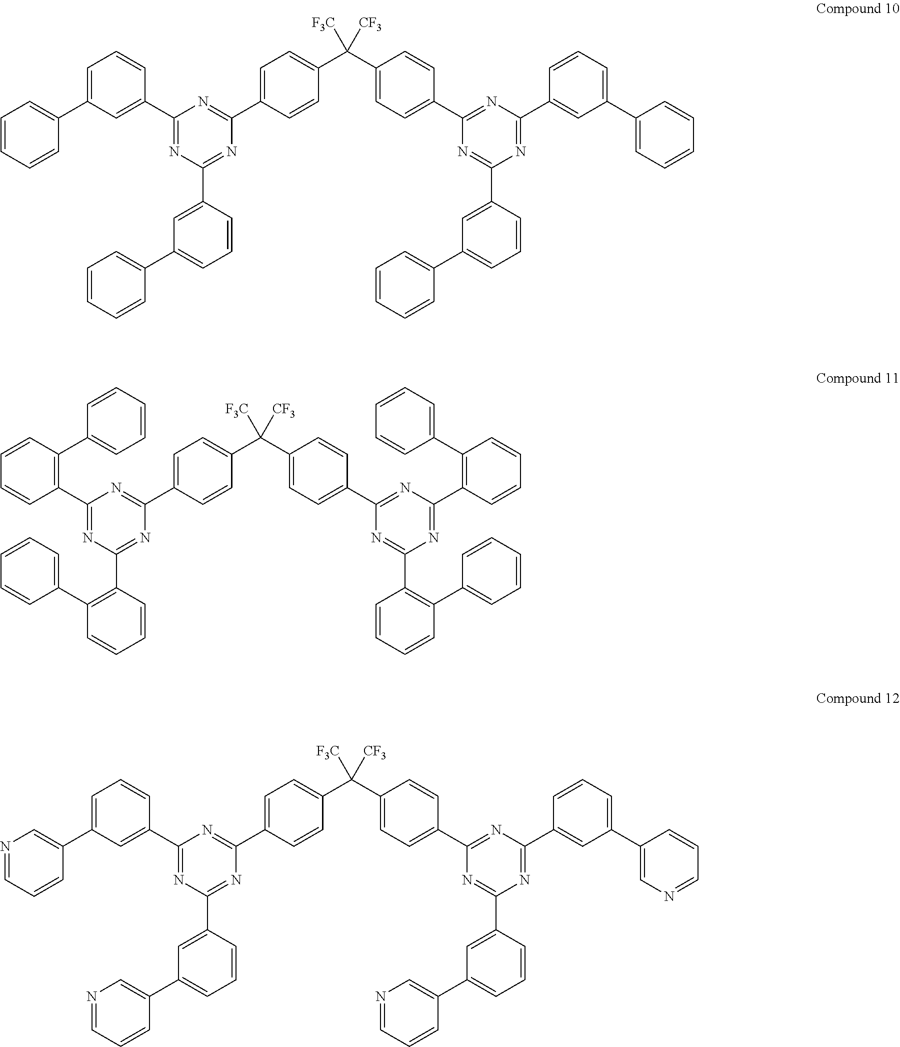 [SMILES: FC(F)(F)C(c1ccc(-c2nc(-c3cccc(-c4ccccc4)c3)nc(-c3cccc(-c4ccccc4)c3)n2)cc1)(c1ccc(-c2nc(-c3cccc(-c4ccccc4)c3)nc(-c3cccc(-c4ccccc4)c3)n2)cc1)C(F)(F)F ; FC(F)(F)C(c1ccc(-c2nc(-c3ccccc3-c3ccccc3)nc(-c3ccccc3-c3ccccc3)n2)cc1)(c1ccc(-c2nc(-c3ccccc3-c3ccccc3)nc(-c3ccccc3-c3ccccc3)n2)cc1)C(F)(F)F ; FC(F)(F)C(c1ccc(-c2nc(-c3cccc(-c4cccnc4)c3)nc(-c3cccc(-c4cccnc4)c3)n2)cc1)(c1ccc(-c2nc(-c3cccc(-c4cccnc4)c3)nc(-c3cccc(-c4cccnc4)c3)n2)cc1)C(F)(F)F]

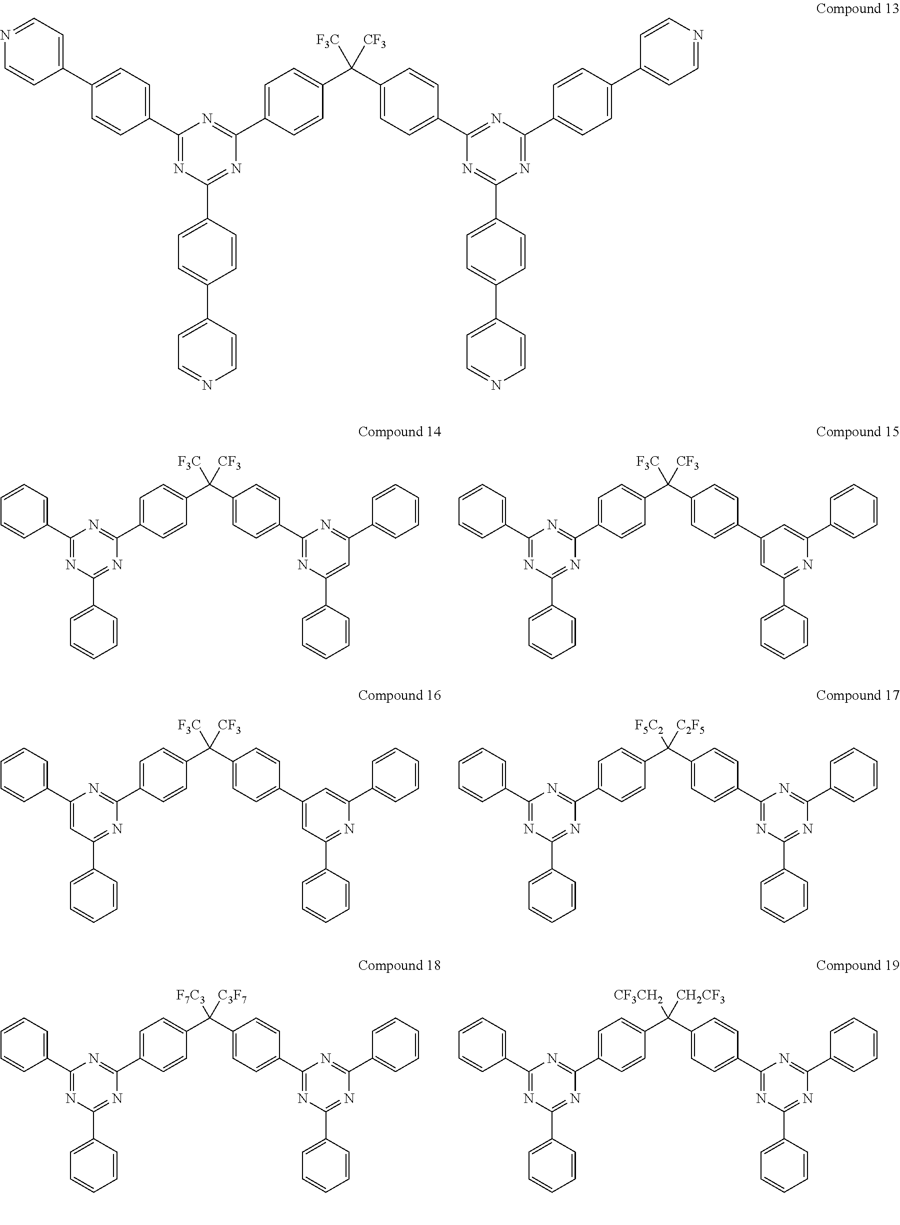

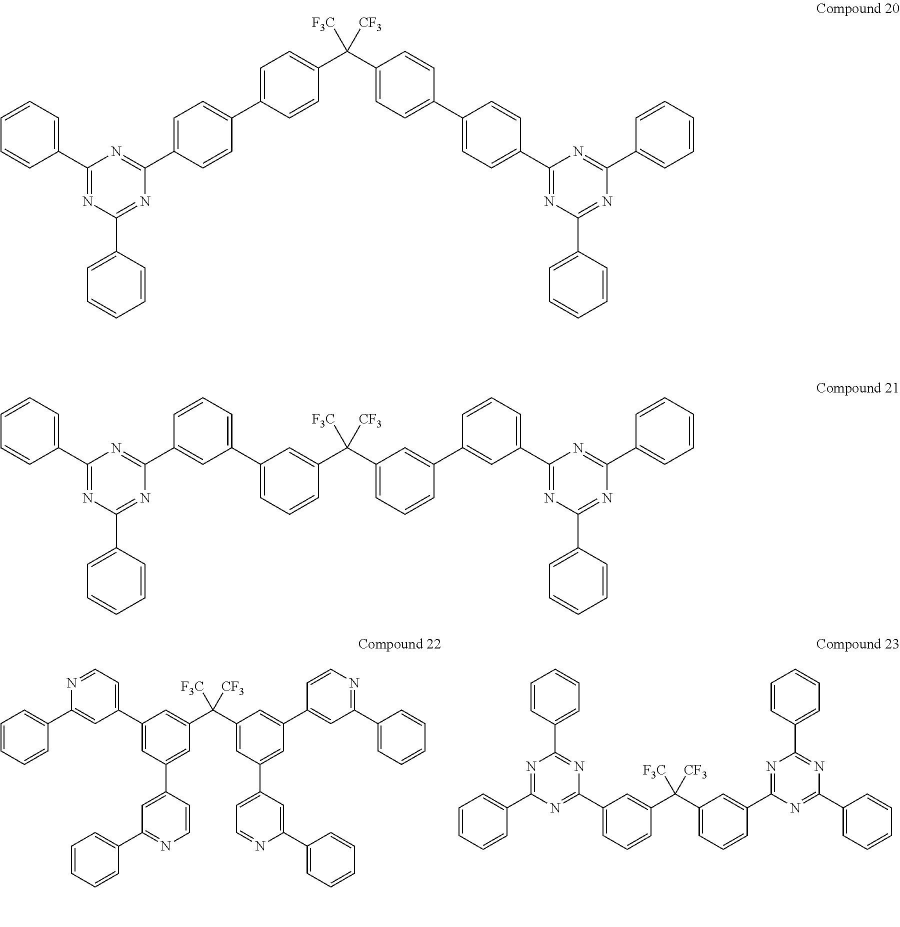 [SMILES: FC(F)(F)C(c1ccc(-c2ccc(-c3nc(-c4ccccc4)nc(-c4ccccc4)n3)cc2)cc1)(c1ccc(-c2ccc(-c3nc(-c4ccccc4)nc(-c4ccccc4)n3)cc2)cc1)C(F)(F)F ; FC(F)(F)C(c1cccc(-c2cccc(-c3nc(-c4ccccc4)nc(-c4ccccc4)n3)c2)c1)(c1cccc(-c2cccc(-c3nc(-c4ccccc4)nc(-c4ccccc4)n3)c2)c1)C(F)(F)F ; FC(F)(F)C(c1cc(-c2ccnc(-c3ccccc3)c2)cc(-c2ccnc(-c3ccccc3)c2)c1)(c1cc(-c2ccnc(-c3ccccc3)c2)cc(-c2ccnc(-c3ccccc3)c2)c1)C(F)(F)F ; FC(F)(F)C(c1cccc(-c2nc(-c3ccccc3)nc(-c3ccccc3)n2)c1)(c1cccc(-c2nc(-c3ccccc3)nc(-c3ccccc3)n2)c1)C(F)(F)F]

The molecular weight of the compound represented by the formula (1) is, for example, in the case where an organic layer containing the compound represented by the formula (1) is intended to be formed according to a vapor deposition method and used in devices, preferably 1500 or less, more preferably 1200 or less, even more preferably 1000 or less, and furthermore preferably 900 or less. The lower limit of the molecular weight is the smallest molecular weight that the formula (1) can take.

Irrespective of the molecular weight thereof, the compound represented by the formula (1) may be formed into a film according to a coating method. When a coating method is employed, even a compound having a relatively large molecular weight can be formed into a film.

Applying the present invention, it is considered to use a compound containing plural structures represented by the formula (1) in the molecule as a charge transport material.

For example, it is considered that a polymerizable group is previously introduced into a structure represented by the formula (1) and the polymerizable group is polymerized to give a polymer, and the polymer is used as a charge transport material. Specifically, a monomer containing a polymerizable functional group in any of R¹, R², Ar¹, Ar², A¹ and A² in the formula (1) is prepared, and this is homo-polymerized or copolymerized with any other monomer to give a polymer having a recurring unit, and the polymer can be used as a material for a charge transport material. Alternatively, compounds each having a structure represented by the formula (1) are coupled to give a dimer or a trimer, and it can be used as a charge transport material.

Examples of the polymer having a recurring unit containing a structure represented by the formula (1) include polymers containing a structure represented by the following formula (11) or (12).

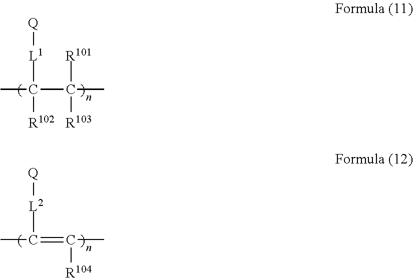

In the formula (11) or (12), Q represents a group containing a structure represented by the formula (1), and L¹ and L² each represent a linking group. The carbon number of the linking group is preferably 0 to 20, more preferably 1 to 15, even more preferably 2 to 10. Preferably, the linking group has a structure represented by —X¹¹-L¹¹-. Here, X¹¹ represents an oxygen atom or a sulfur atom and is preferably an oxygen atom. L¹¹ represents a linking group, and is preferably a substituted or unsubstituted alkylene group, or a substituted or unsubstituted arylene group, more preferably a substituted or unsubstituted alkylene group having 1 to 10 carbon atoms, or a substituted or unsubstituted phenylene group.

In the formula (11) or (12), R¹⁰¹, R¹⁰², R¹⁰³ and R¹⁰⁴ each independently represent a substituent. Preferably, the substituent is a substituted or unsubstituted alkyl group having 1 to 6 carbon atoms, a substituted or unsubstituted alkoxy group having 1 to 6 carbon atoms, or a halogen atom, more preferably an unsubstituted alkyl group having 1 to 3 carbon atoms, an unsubstituted alkoxy group having 1 to 3 carbon atoms, a fluorine atom, or a chlorine atom, and even more preferably an unsubstituted alkyl group having 1 to 3 carbon atoms, or an unsubstituted alkoxy group having 1 to 3 carbon atoms.

The linking group represented by L¹ and L² may bond to any of R¹, R², Ar¹, Ar², A¹ and A² in the structure of the formula (1) constituting Q. Two or more linking groups may bond to one Q to form a crosslinked structure or a network structure.

Specific examples of the structure of the recurring unit include structures represented by the following formulae (13) to (16).

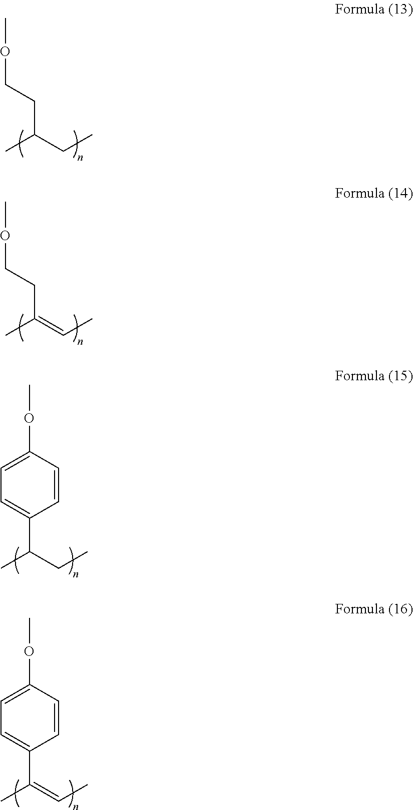

The polymer having the recurring unit containing the structure represented by any of the formulae (13) to (16) may be synthesized in such a manner that a hydroxyl group is introduced to any of R¹, R², Ar¹, Ar², A¹ and A² in the structure represented by the formula (1), and the hydroxyl group as a linker is reacted with the following compound to introduce a polymerizable group thereinto, followed by polymerizing the polymerizable group.

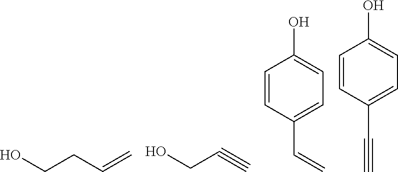

The polymer containing the structure represented by the formula (1) in the molecule may be a polymer containing only a recurring unit having the structure represented by the formula (1), or a polymer further containing a recurring unit having another structure. The recurring unit having the structure represented by the formula (1) contained in the polymer may be only one kind or two or more kinds. Examples of the recurring unit that does not have the structure represented by the formula (1) include a recurring unit derived from a monomer that is used for ordinary copolymerization. Examples of the recurring unit include a recurring unit derived from a monomer having an ethylenic unsaturated bond, such as ethylene and styrene.

### [Synthesis Method for Compound Represented by Formula (1)]

The compound represented by the formula (1) can be synthesized by combining known reactions. For example, a compound of the formula (1) where Ar¹ and Ar² each are a benzene ring, and A¹ and A² each are a group represented by the formula (2) can be synthesized by synthesizing an intermediate b′ according to the following reaction scheme 1, and then coupling the intermediate b′ with a precursor corresponding to a partial structure (a group bonding to L¹⁹) of the formula (2) according to coupling reaction.

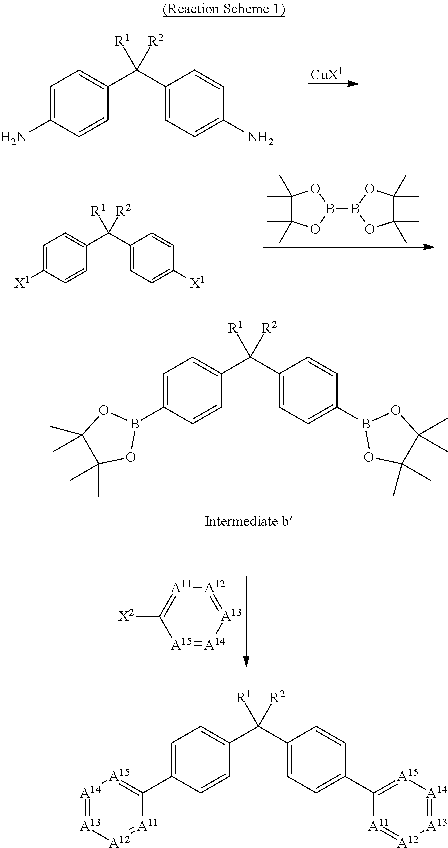

Regarding the description of R¹ and R² in the above reaction scheme 1, reference may be made to the corresponding description of the formula (1), and regarding the description of A¹¹ to A¹⁵, reference may be made to the corresponding description of the formula (2). X¹ and X² each independently represent a halogen atom, including a fluorine atom, a chlorine atom, a bromine atom and an iodine atom. X¹ is preferably a bromine atom, and X² is preferably a chlorine atom.

A known coupling reaction is applied to the above reaction, for which any known reaction condition can be appropriately selected and used. Regarding the details of the above reaction, reference may be made to Synthesis Examples given hereinunder. The compound represented by the formula (1) can also be synthesized by combining any other known synthesis reactions.

### [Organic Light-Emitting Device]

The compound represented by the formula (1) of the present invention is useful as a charge transport material for organic light-emitting devices. Accordingly, the compound represented by the formula (1) of the present invention can be effectively used as a host material in a light-emitting layer, an electron transport material in an electron transport layer of an organic light-emitting device, or the like. With that, the present invention can realize an organic light-emitting device having a low drive voltage, an organic light-emitting device having a high light emission efficiency, and an organic light-emitting device having a long lifetime. Above all, the compound having a lowest excited triplet energy level (En) of 2.90 eV or more, preferably 2.95 eV or more, more preferably 3.00 eV or more is useful as a material for an organic light-emitting device having a short emission wavelength. For example, the compound is useful as a material for an organic light-emitting device having a maximum emission wavelength of 360 to 550 nm, especially 360 to 495 nm.

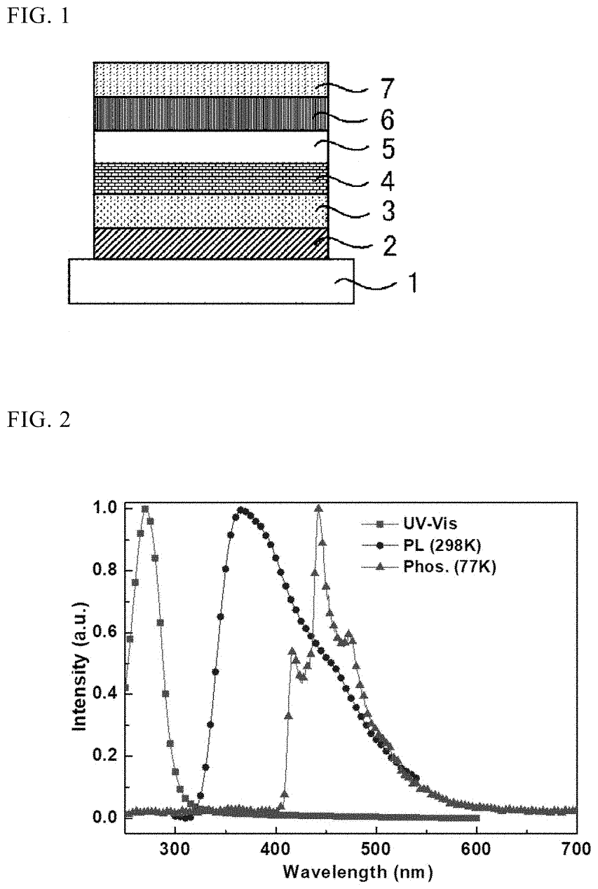

Using the compound represented by the formula (1) of the present invention as a charge transport material, excellent organic light-emitting devices such as an organic photoluminescent device (organic PL device) and an organic electroluminescent device (organic EL device) can be provided. An organic photoluminescent device has a structure where at least a light-emitting layer is formed on a substrate. An organic electroluminescent device has a structure including at least an anode, a cathode and an organic layer formed between the anode and the cathode. The organic layer contains at least a light-emitting layer, and may be formed of a light-emitting layer alone, or may has one or more other organic layers in addition to a light-emitting layer. The other organic layers include a hole transport layer, a hole injection layer, an electron blocking layer, a hole blocking layer, an electron injection layer, an electron transport layer, and an exciton blocking layer. The hole transport layer may be a hole injection transport layer having a hole injection function, and the electron transport layer may be an electron injection transport layer having an electron injection function. A configuration example of an organic electroluminescent device is shown in FIG. 1. In FIG. 1, 1 is a substrate, 2 is an anode, 3 is a hole injection layer, 4 is a hole transport layer, 5 is a light-emitting layer, 6 is an electron transport layer, and 7 is a cathode.

In the following, the constituent members and the layers of the organic electroluminescent device are described. The compound represented by the formula (1) is contained in at least one layer formed between an anode and a cathode in an organic electroluminescent device. The description of the substrate and the light-emitting layer given below may apply to the substrate and the light-emitting layer of an organic photoluminescent device.

### (Substrate)

The organic electroluminescent device of the invention is preferably supported by a substrate. The substrate is not particularly limited and may be those that have been commonly used in an organic electroluminescent device, and examples thereof used include those formed of glass, transparent plastics, quartz and silicon.

### (Anode)

The anode of the organic electroluminescent device used is preferably formed of, as an electrode material, a metal, an alloy, or an electroconductive compound each having a large work function (4 eV or more), or a mixture thereof. Specific examples of the electrode material include a metal, such as Au, and an electroconductive transparent material, such as CuI, indium tin oxide (ITO), SnO₂ and ZnO. A material that is amorphous and is capable of forming a transparent electroconductive film, such as IDIXO (In₂O₃—ZnO), may also be used. The anode may be formed in such a manner that the electrode material is formed into a thin film by such a method as vapor deposition or sputtering, and the film is patterned into a desired pattern by a photolithography method, or in the case where the pattern may not require high accuracy (for example, approximately 100 μm or more), the pattern may be formed with a mask having a desired shape on vapor deposition or sputtering of the electrode material. In alternative, in the case where a material capable of being coated, such as an organic electroconductive compound, is used, a wet film forming method, such as a printing method and a coating method, may be used. In the case where emitted light is to be taken out through the anode, the anode preferably has a transmittance of more than 10%, and the anode preferably has a sheet resistance of several hundred Ω/sq or less. The thickness of the anode may be generally selected from a range of from 10 to 1,000 nm, and preferably from 10 to 200 nm, while depending on the material used.

### (Cathode)

The cathode is preferably formed of, as an electrode material, a metal (which is referred to as an electron injection metal), an alloy, or an electroconductive compound, having a small work function (4 eV or less), or a mixture thereof. Specific examples of the electrode material include sodium, a sodium-potassium alloy, magnesium, lithium, a magnesium-cupper mixture, a magnesium-silver mixture, a magnesium-aluminum mixture, a magnesium-indium mixture, an aluminum-aluminum oxide (Al₂O₃) mixture, indium, a lithium-aluminum mixture, and a rare earth metal. Among these, a mixture of an electron injection metal and a second metal that is a stable metal having a larger work function than the electron injection metal, for example, a magnesium-silver mixture, a magnesium-aluminum mixture, a magnesium-indium mixture, an aluminum-aluminum oxide (Al₂O₃) mixture, a lithium-aluminum mixture, and aluminum, is preferred from the standpoint of the electron injection property and the durability against oxidation and the like. The cathode may be produced by forming the electrode material into a thin film by such a method as vapor deposition or sputtering. The cathode preferably has a sheet resistance of several hundred Ω/sq or less, and the thickness thereof may be generally selected from a range of from 10 nm to 5 μm, and preferably from 50 to 200 nm. For transmitting the emitted light, any one of the anode and the cathode of the organic electroluminescent device is preferably transparent or translucent, thereby enhancing the light emission luminance.

The cathode may be formed with the electroconductive transparent materials described for the anode, thereby forming a transparent or translucent cathode, and by applying the cathode, a device having an anode and a cathode, both of which have transmittance, may be produced.

### (Light-Emitting Layer)

The light-emitting layer is a layer in which holes and electrons injected from an anode and a cathode are recombined to give excitons for light emission, and the layer may be formed of a light-emitting material alone, or may contain a light-emitting material and a host material. As the light-emitting material, any known one can be used, and may be any of a fluorescent material, a delayed fluorescent material or a phosphorescent material, but is preferably a delayed fluorescent material as achieving a high light emission efficiency.

As the host material, one or more selected from a group of the compounds of the present invention represented by the formula (1) can be used. Among the group of the compounds of the formula (1), preferred for use as the host material is a compound such that at least any one of the lowest excited singlet energy level and the lowest excited triplet energy level thereof is higher than that of the light-emitting material, and more preferred is a compound such that both the lowest excited singlet energy level and the lowest excited triplet energy level thereof are higher than those of the light-emitting material. With that, the singlet exciton and the triplet exciton formed in the light-emitting material can be confined to the molecule of the light-emitting material of the present invention to sufficiently derive the light emission efficiency thereof. The light emission may be any of fluorescent emission, delayed fluorescent emission or phosphorescent emission, and may contain two or more types of light emission. In addition, a part of light emission may be partially from a host material.

The content of the light-emitting material in the light-emitting layer is preferably less than 50% by weight. Further, the upper limit of the content of the light-emitting material is preferably less than 30/by weight, and the upper limit of the content may be, for example, less than 20% by weight, less than 10% by weight, less than 5% by weight, less than 3% by weight, less than 1% by weight, or less than 0.5% by weight. Preferably, the lower limit is 0.001% by weight or more, and may be, for example, more than 0.01% by weight, more than 0.1% by weight, more than 0.5% by weight, or more than 1% by weight.

The light-emitting layer preferably contains a compound such that a difference ΔE_{ST} between the lowest excited singlet energy level and the lowest excited triplet energy level thereof is 0.3 eV or less. A compound having ΔE_{ST} of less than 0.3 eV can readily undergo reverse intersystem crossing from the excited triplet state to the excited singlet state and therefore can be effectively used as a material for converting excited triplet energy to excited singlet energy. Specifically, the light-emitting layer can contain a compound having ΔE_{ST} of less than 0.3 eV as a light-emitting material. In this case, the compound having ΔE_{ST} of less than 0.3 eV functions as a delayed fluorescent material that emits delayed fluorescence, and accordingly can achieve a high light emission efficiency. The principle of the delayed fluorescent material to achieve a high light emission efficiency is as follows.

Specifically, in an organic electroluminescent device, a carrier is injected into a light-emitting material through both a positive electrode and a negative electrode to form a light-emitting material in an excited state to attain light emission. In general, in the case of a carrier injection-type organic electroluminescent device, 25% of the formed excitons are excited to be in a excited singlet state, and the remaining 75% thereof are excited in an excited triplet state. Accordingly, phosphorescence to be emitted from the excited triplet state can be utilized at a higher energy utilization efficiency. However, the excited triplet state has a short lifetime, therefore often causing energy deactivation owing to saturation of the excited state or interaction with excitons in a excited triplet state, and consequently, in general, a phosphorescence quantum yield is not high in many cases. On the other hand, a delayed fluorescent material emits fluorescence after energy transition to an excited triplet state by intersystem crossing or the like followed by reverse intersystem crossing to an excited singlet state owing to triplet-triplet annihilation or heat energy absorption. It is considered that among the materials, a thermal activation type delayed fluorescent material emitting light through absorption of thermal energy is particularly useful for an organic electroluminescent device. In the case where a delayed fluorescent material is used in an organic electroluminescent device, the excitons in the excited singlet state normally emit fluorescent light. On the other hand, the excitons in the excited triplet state emit fluorescent light through intersystem crossing to the excited singlet state by absorbing the heat generated by the device. At this time, the light emitted through reverse intersystem crossing from the excited triplet state to the excited singlet state has the same wavelength as fluorescent light since it is light emission from the excited singlet state, but has a longer lifetime (light emission lifetime) than the normal fluorescent light or phosphorescence, and thus the light is observed as fluorescent light that is delayed from the normal fluorescent light. The light may be defined as delayed fluorescent light. The use of the thermal activation type exciton transfer mechanism may raise the proportion of the compound in the excited singlet state, which is generally formed in a proportion only of 25%, to 25% or more through the absorption of the thermal energy after the carrier injection. A compound that emits strong fluorescent light and delayed fluorescent light at a low temperature of lower than 100° C. undergoes the intersystem crossing from the excited triplet state to the excited singlet state sufficiently with the heat of the device, thereby emitting delayed fluorescent light, and thus the use of the compound may drastically enhance the light emission efficiency.

The light-emitting layer may contain a compound having ΔE_{ST} of 0.3 eV or less as an assist dopant. Here, an assist dopant is a material that is used as combined with a host material and a light-emitting material to promote light emission from the light-emitting material. When the light-emitting layer contains a compound having ΔE_{ST} of 0.3 eV or less as an assist dopant, the excited triplet energy generated in the host material owing to carrier recombination in the light-emitting layer and the excited triplet energy generated in the assist dopant can be converted into excited singlet energy through reverse intersystem crossing and, consequently, the excited singlet energy can be effectively utilized for fluorescence emission from the light-emitting material. In the system using such an assist dopant, preferably a fluorescent material or a delayed fluorescent material capable of emitting light through radiation deactivation from the excited singlet state is used as a light-emitting material. As a host material, one or more selected from a group of compounds of the present invention represented by the formula (1) can be used. Preferably, the assist dopant has ΔE_{ST} of 0.3 eV or less, has a higher lowest excited singlet energy level than the light-emitting material and has a lower lowest excited singlet energy level than the host material. Accordingly, the excited singlet energy generated in the host material can readily move toward the assist dopant and the light-emitting material, and the excited singlet energy generated in the assist dopant, and the excited singlet energy transferred from the host material to the assist dopant can readily move toward the light-emitting material. As a result, the light-emitting material in an excited singlet state can be efficiently formed to achieve a high light emission efficiency. Further, more preferably, the assist dopant has a lower lowest excited triplet energy level than the host material. Accordingly, the excited triplet energy generated in the host material readily moves toward the assist dopant and is converted into excited singlet energy through reverse intersystem crossing in the assist dopant. As a result of transfer of the excited singlet energy of the assist dopant to the light-emitting material, the light-emitting material in an excited singlet state can be further more efficiently formed to achieve an extremely high light emission efficiency.

In a system where the light-emitting layer contains a light-emitting material, a host material and an assist dopant, the content of the assist dopant in the light-emitting layer is preferably smaller than the content of the host material and larger than the content of the light-emitting material, that is, the three preferably satisfy a relationship of “content of light-emitting material<content of assist dopant<content of host material”. Specifically, in this embodiment, the content of the assist dopant in the light-emitting layer is preferably less than 50% by weight. Further, the upper limit of the content of the assist dopant is preferably less than 40% by weight, and the upper limit of the content can be, for example, less than 30% by weight, or less than 20% by weight, or less than 10% by weight. The lower limit is preferably 0.1% by weight or more, for example, more than 1% by weight, or more than 3% by weight.

In the case where a compound represented by the formula (1) is used in a light-emitting layer, the content of the compound represented by the formula (1) in the light-emitting layer is preferably 50% by weight or more, more preferably more than 60% by weight, and may be more than 70% by weight, more than 80% by weight, more than 90% by weight, more than 95% by weight, more than 97% by weight, more than 99% by weight, or more than 99.5% by weight, in any of a system using a light-emitting material and a host material, or a system using a light-emitting material, an assist dopant and a host material. The upper limit of the content is preferably 99.999% by weight or less in a system using a light-emitting material and a host material, and is preferably 990.899% by weight or less in a system containing a light-emitting material, an assist dopant and a host material.

In the case where the light-emitting layer contains a compound having ΔE_{ST} of 0.3 eV or less, ΔE_{ST} thereof is preferably 0.2 eV or less, more preferably 0.1 eV or less.

Here, the lowest excited singlet energy level (E_{S1}) and the lowest excited triplet energy level (E_{T1}) of the compound can be calculated according to the following method, and the difference ΔE_{ST} between the lowest excited singlet energy level (E_{S1}) and the lowest excited triplet energy level (E_{T1}) can be calculated according to an equation, ΔE_{ST}=E_{S1}−E_{T1}.

### (1) Lowest Excited Singlet Energy Level (E_{S1})

The compound to be analyzed and mCP were co-deposited through vapor evaporation on an Si substrate so that the concentration of the compound could be 6% by weight to thereby prepare a sample having a thickness of 100 nm on the Si substrate. Alternatively, a toluene solution of the compound to be analyzed is prepared to have a concentration of 1×10⁻⁵ mol/L. The fluorescence spectrum of the sample is measured at room temperature (300 K). Specifically, the emitted light is integrated from immediately after irradiation with the excited light until 100 nanoseconds after the irradiation to achieve a fluorescence spectrum on a graph where the vertical axis indicates an emission intensity and the horizontal axis indicates a wavelength. A tangent line is drawn to the rising of the photoluminescence spectrum on the short wavelength side, and the wavelength value ledge [nm] at the intersection between the tangent line and the horizontal axis is read. The wavelength value is converted into an energy value according to the following conversion expression to calculate E_{S1}.

E_{S1} [eV]=1239.85/λedge Conversion Expression:

For the measurement of the photoluminescence spectrum, a nitrogen laser (MNL200, from Lasertechnik Berlin) can be used as an excitation light source, and a streak camera (C4334, from Hamamatsu Photonics) can be used as a detector.

### (2) Lowest Excited Triplet Energy Level (E_{T1})

The same sample as that for measurement of the lowest excited singlet energy level E_{S1} is cooled to 5 [K], and the sample for phosphorescence measurement is irradiated with excitation light (337 nm), and using a streak camera, the phosphorescence intensity thereof is measured. The emission from 1 millisecond after irradiation with the excitation light until 10 milliseconds after the irradiation is integrated to achieve a phosphorescence spectrum on a graph where the vertical axis indicates an emission intensity and the horizontal axis indicates a wavelength. A tangent line is drawn to the rising of the phosphorescence spectrum on the short wavelength side, and the wavelength value ledge [nm] at the intersection between the tangent line and the horizontal axis is read. The wavelength value is converted into an energy value according to the following conversion expression to calculate E_{T1}.

E_{T1} [eV]=1239.85/λedge Conversion Expression:

The tangent line to the rising of the phosphorescence spectrum on the short wavelength side is drawn as follows. While moving on the spectral curve from the short wavelength side of the phosphorescence spectrum toward the maximum value on the shortest wavelength side among the maximum values of the spectrum, a tangent line at each point on the curve toward the long wavelength side is taken into consideration. With rising the curve (that is, with increase in the vertical axis), the inclination of the tangent line increases. The tangent line drawn at the point at which the inclination value has a maximum value is referred to as the tangent line to the rising on the short wavelength side of the phosphorescence spectrum.

The maximum point having a peak intensity of 10% or less of the maximum peak intensity of the spectrum is not included in the maximum value on the above-mentioned shortest wavelength side, and the tangent line drawn at the point which is closest to the maximum value on the shortest wavelength side and at which the inclination value has a maximum value is referred to as the tangent line to the rising on the short wavelength side of the phosphorescence spectrum.

### (Injection Layer)

The injection layer is a layer that is provided between the electrode and the organic layer, for decreasing the driving voltage and enhancing the light emission luminance, and includes a hole injection layer and an electron injection layer, which may be provided between the anode and the light-emitting layer or the hole transport layer and between the cathode and the light emitting layer or the electron transport layer. The injection layer may be provided depending on necessity.

### (Blocking Layer)

The blocking layer is a layer that is capable of inhibiting charges (electrons or holes) and/or excitons present in the light-emitting layer from being diffused outside the light-emitting layer. The electron blocking layer may be disposed between the light-emitting layer and the hole transport layer, and inhibits electrons from passing through the light-emitting layer toward the hole transport layer. Similarly, the hole blocking layer may be disposed between the light-emitting layer and the electron transport layer, and inhibits holes from passing through the light-emitting layer toward the electron transport layer. The blocking layer may also be used for inhibiting excitons from being diffused outside the light-emitting layer. Thus, the electron blocking layer and the hole blocking layer each may also have a function as an exciton blocking layer. The term “the electron blocking layer” or “the exciton blocking layer” referred to herein is intended to include a layer that has both the functions of an electron blocking layer and an exciton blocking layer by one layer.

### (Hole Blocking Layer)

The hole blocking layer has the function of an electron transport layer in a broad sense. The hole blocking layer has a function of inhibiting holes from reaching the electron transport layer while transporting electrons, and thereby enhances the recombination probability of electrons and holes in the light-emitting layer. As the material for the hole blocking layer, the material for the electron transport layer to be mentioned below may be used optionally.

### (Electron Blocking Layer)

The electron blocking layer has the function of transporting holes in a broad sense. The electron blocking layer has a function of inhibiting electrons from reaching the hole transport layer while transporting holes, and thereby enhances the recombination probability of electrons and holes in the light-emitting layer.

### (Exciton Blocking Layer)

The exciton blocking layer is a layer for inhibiting excitons generated through recombination of holes and electrons in the light-emitting layer from being diffused to the charge transporting layer, and the use of the layer inserted enables effective confinement of excitons in the light-emitting layer, and thereby enhances the light emission efficiency of the device. The exciton blocking layer may be inserted adjacent to the light-emitting layer on any of the side of the anode and the side of the cathode, and on both the sides. Specifically, in the case where the exciton blocking layer is present on the side of the anode, the layer may be inserted between the hole transport layer and the light-emitting layer and adjacent to the light-emitting layer, and in the case where the layer is inserted on the side of the cathode, the layer may be inserted between the light-emitting layer and the cathode and adjacent to the light-emitting layer. Between the anode and the exciton blocking layer that is adjacent to the light-emitting layer on the side of the anode, a hole injection layer, an electron blocking layer and the like may be provided, and between the cathode and the exciton blocking layer that is adjacent to the light-emitting layer on the side of the cathode, an electron injection layer, an electron transport layer, a hole blocking layer and the like may be provided. In the case where the blocking layer is provided, preferably, at least one of the excited singlet energy and the excited triplet energy of the material used as the blocking layer is higher than the excited singlet energy and the excited triplet energy, of the light-emitting material.

### (Hole Transport Layer)

The hole transport layer is formed of a hole transport material having a function of transporting holes, and the hole transport layer may be provided as a single layer or plural layers.

The hole transport material has one of injection or transporting property of holes and blocking property of electrons, and may be any of an organic material and an inorganic material. Examples of known hole transport materials that may be used herein include a triazole derivative, an oxadiazole derivative, an imidazole derivative, a carbazole derivative, an indolocarbazole derivative, a polyarylalkane derivative, a pyrazoline derivative, a pyrazolone derivative, a phenylenediamine derivative, an arylamine derivative, an amino-substituted chalcone derivative, an oxazole derivative, a styrylanthracene derivative, a fluorenone derivative, a hydrazone derivative, a stilbene derivative, a silazane derivative, an aniline copolymer and an electroconductive polymer oligomer, particularly a thiophene oligomer. Among these, a porphyrin compound, an aromatic tertiary amine compound and a styrylamine compound are preferably used, and an aromatic tertiary amine compound is more preferably used.

### (Electron Transport Layer)

The electron transport layer is formed of a material having a function of transporting electrons, and the electron transport layer may be a single layer or may be formed of plural layers.

The electron transport material (often also acting as a hole blocking material) may have a function of transmitting the electrons injected from a cathode to a light-emitting layer. As the electron transport material, compounds represented by the formula (1) can be used. Other electron transport materials than the compounds represented by the formula (1) that can be used in the electron transport layer include, for example, pyridine derivatives, diazine derivatives, triazine derivatives, nitro-substituted fluorene derivatives, diphenylquinone derivatives, thiopyran dioxide derivatives, carbodiimides, fluorenylidenemethane derivatives, anthraquinodimethane and anthrone derivatives, oxadiazole derivatives, etc. Further, thiadiazole derivatives derived from the above-mentioned oxadiazole derivatives by substituting the oxygen atom in the oxadiazole ring with a sulfur atom, and quinoxaline derivatives having a quinoxaline ring known as an electron-attractive group are also usable as the electron transport material. Further, polymer materials prepared by introducing these materials into the polymer chain, or having these material in the polymer main chain are also usable.

In producing the organic electroluminescent device, the compound represented by the formula (1) may be used not only in one organic layer but also in multiple organic layers. In so doing, the compounds represented by the formula (1) used in different organic layers may be the same as or different from each other. For example, a compound represented by the formula (1) is used in a light-emitting layer, and a compound represented by the formula (1) may also be used in the above-mentioned injection layer, the blocking layer, the hole blocking layer, the electron blocking layer, the exciton blocking layer, the hole transport layer, the electron transport layer, etc. The method for forming these layers is not specifically limited, and the layers may be formed according to any of a dry process or a wet process.

Preferred materials for use for the organic electroluminescent device are concretely exemplified below. However, the materials for use in the present invention are not limitatively interpreted by the following exemplary compounds. Compounds, even though exemplified as materials having a specific function, can also be used as other materials having any other function. R, R′, R₁ to R₁₀ in the structural formulae of the following exemplified compounds each independently represent a hydrogen atom or a substituent. X represents a carbon atom or a hetero atom to form a ring skeleton, n represents an integer of 3 to 5, Y represents a substituent, and m represents an integer of 0 or more.

First, specific examples of compounds usable as a delayed fluorescent material as a light-emitting material or as an assist dopant in a light-emitting layer are shown below.

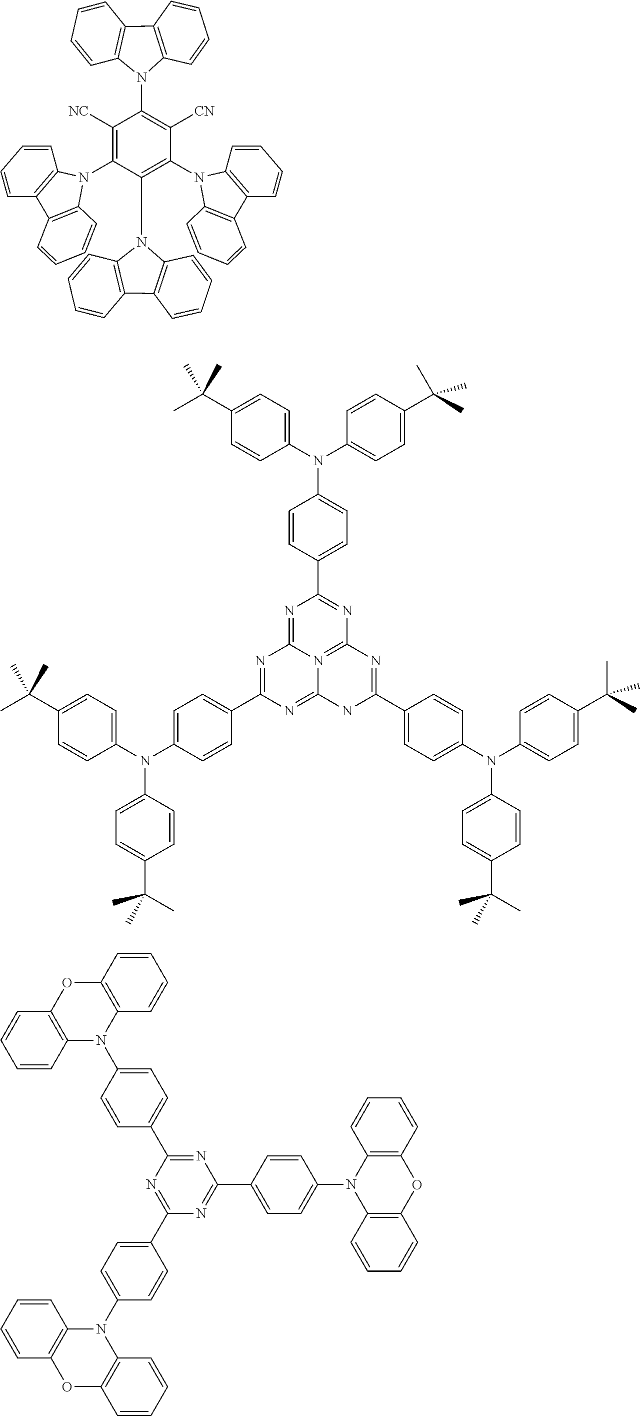

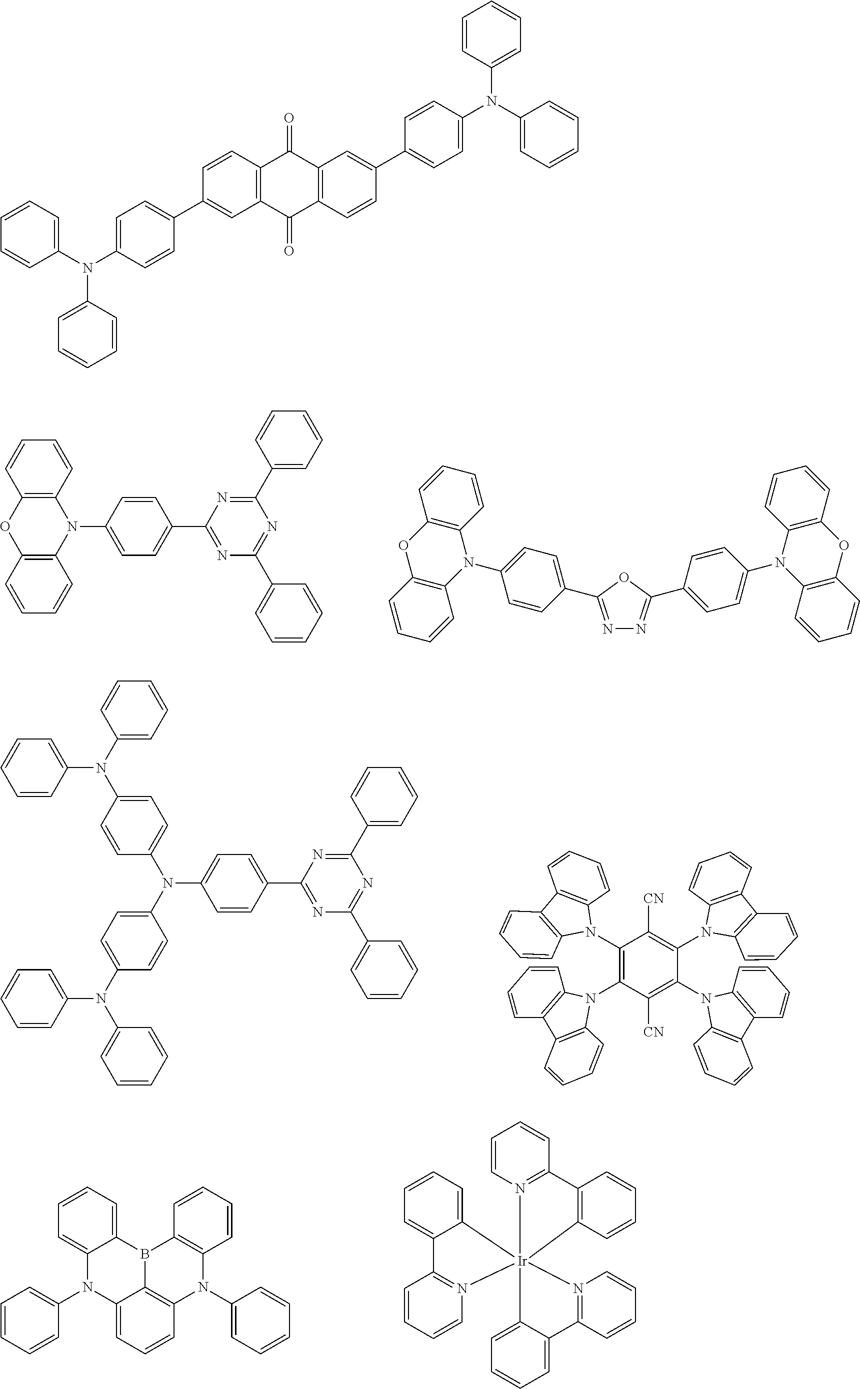

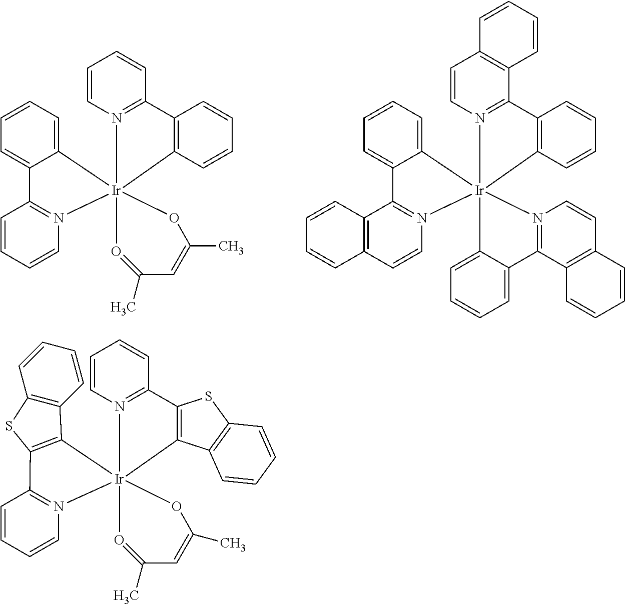

As preferred delayed fluorescent materials, there are mentioned compounds included in formulae described in WO2013/154064, paragraphs 0008 to 0048 and 0095 to 0133; WO2013/011954, paragraphs 0007 to 0047 and 0073 to 0085; WO2013/011955, paragraphs 0007 to 0033 and 0059 to 0066; WO2013/081088, paragraphs 0008 to 0071 and 0118 to 0133; JP 2013-256490 A, paragraphs 0009 to 0046 and 0093 to 0134; JP 2013-116975 A, paragraphs 0008 to 0020 and 0038 to 0040; WO2013/133359, paragraphs 0007 to 0032 and 0079 to 0084; WO2013/161437, paragraphs 0008 to 0054 and 0101-0121; JP 2014-9352 A, paragraphs 0007 to 0041 and 0060 to 0069; JP 2014-9224 A, paragraphs 0008 to 0048 and 0067 to 0076, and especially mentioned are the exemplified compounds capable of emitting delayed fluorescence. In addition, also preferably employable here are light-emitting materials capable of emitting delayed fluorescence, as described in JP 2013-253121 A, WO2013/133359, WO2014/034535, WO2014/115743, WO2014/122895, WO2014/126200, WO2014/136758, WO2014/133121, WO2014/136860, WO2014/196585, WO2014/189122, WO2014/168101, WO2015/008580, WO2014/203840, WO2015/002213, WO2015/016200, WO2015/019725, WO2015/072470, WO2015/108049, WO2015/080182, WO2015/072537, WO2015/080183, JP 2015-129240 A, WO2015/129714, WO2015/129715, WO2015/133501, WO2015/136880, WO2015/137244, WO2015/137202, WO2015/137136, WO2015/146541, and WO2015/159541. These patent publications described in this paragraph are incorporated herein by reference as a part of this description.

Preferred examples of compounds usable as a hole injection material are mentioned below.

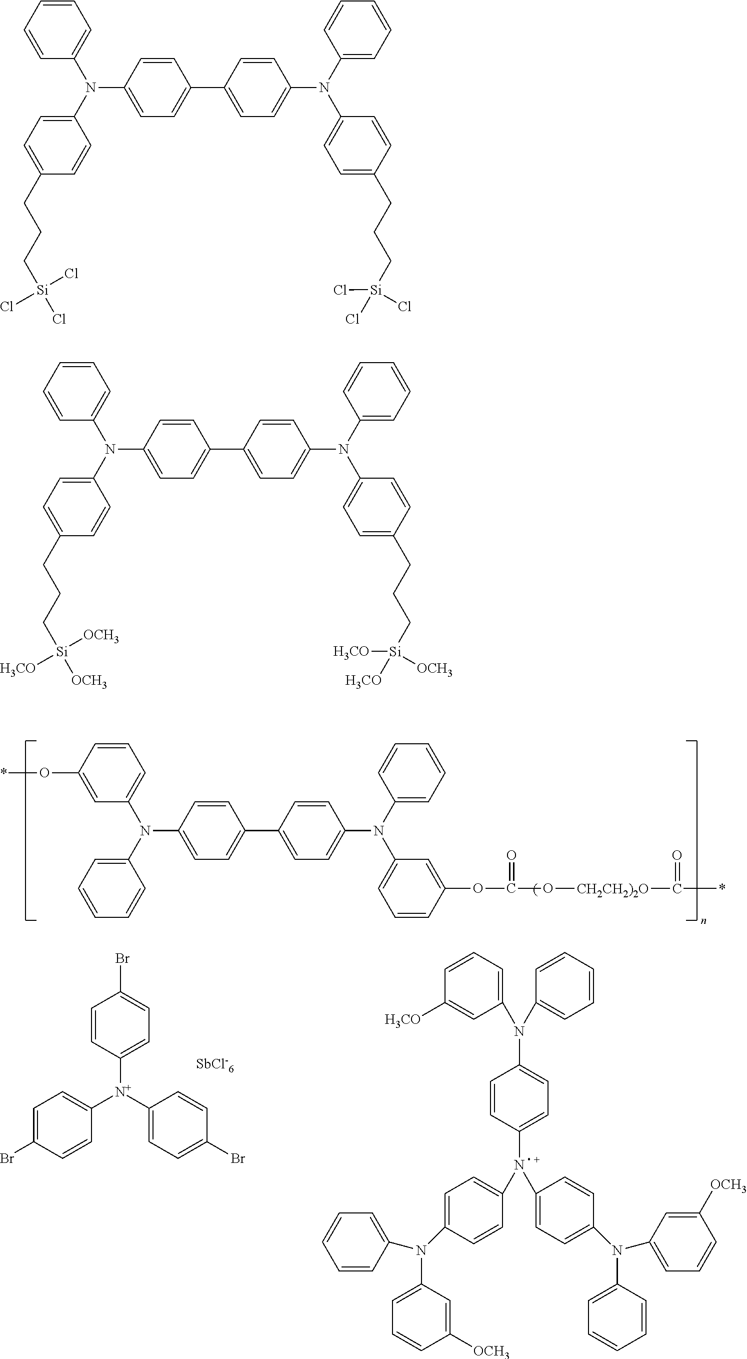

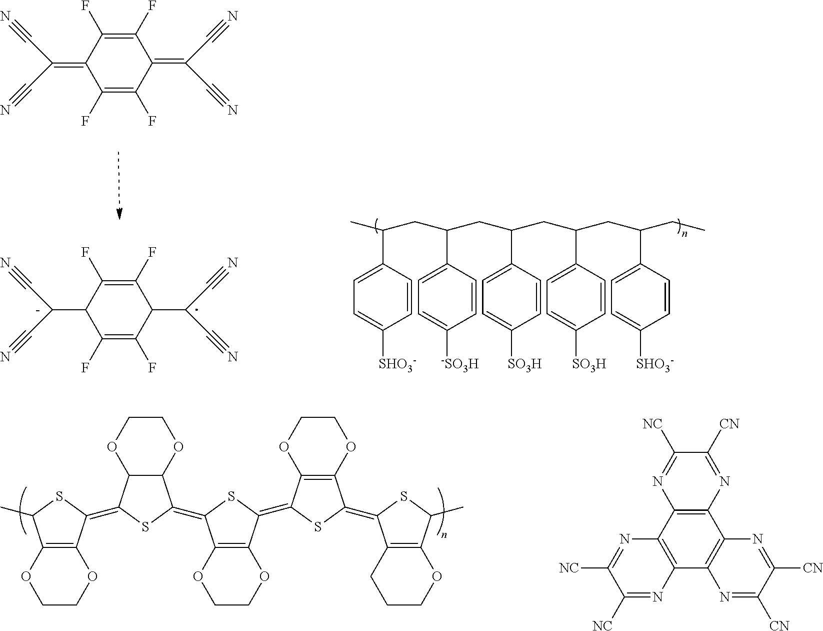

Next, preferred examples of compounds usable as ahole transport material are mentioned below.

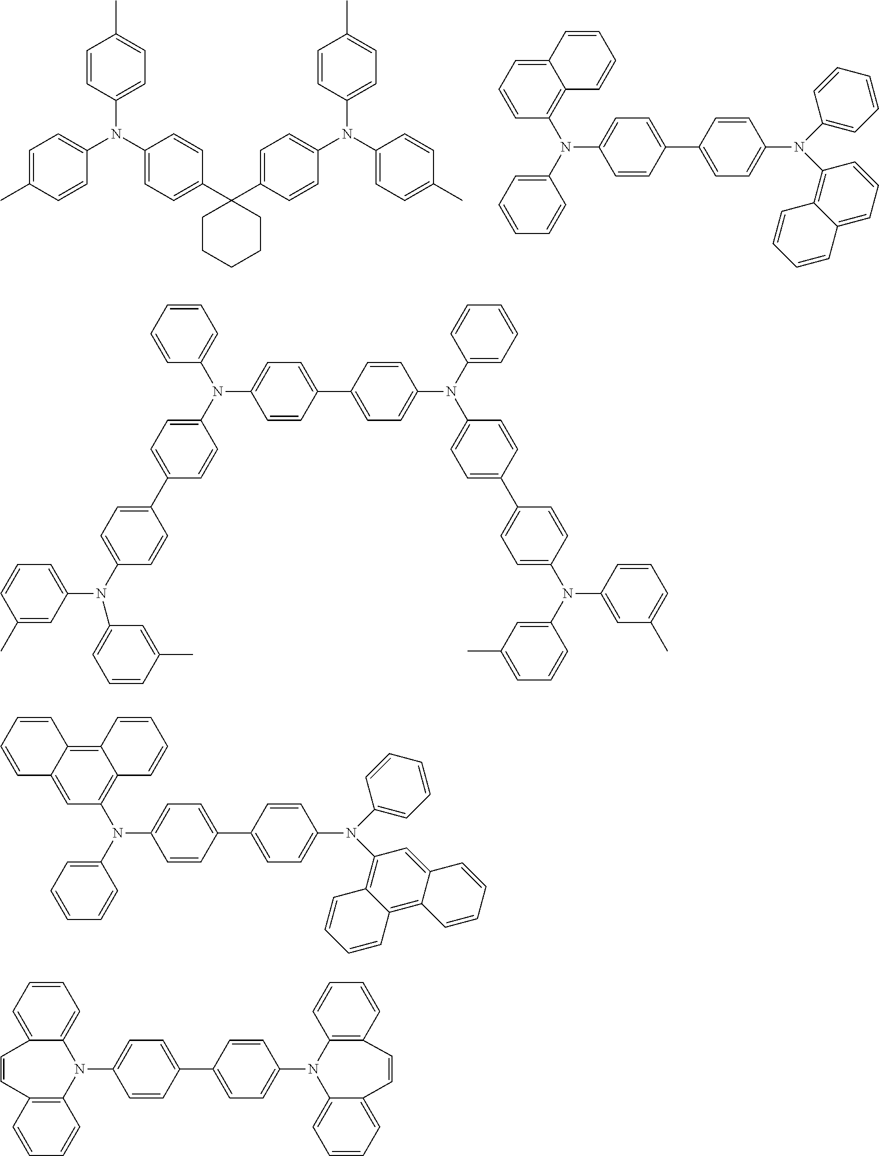

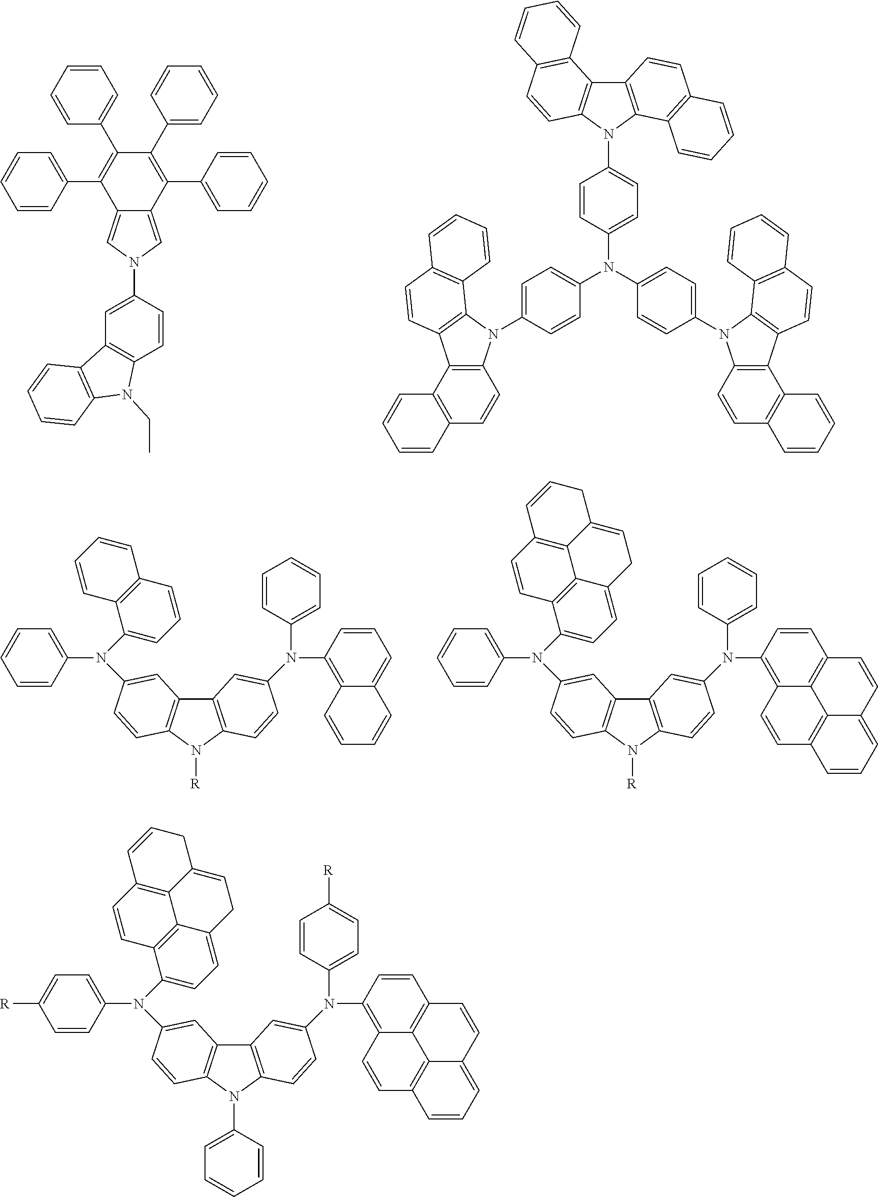

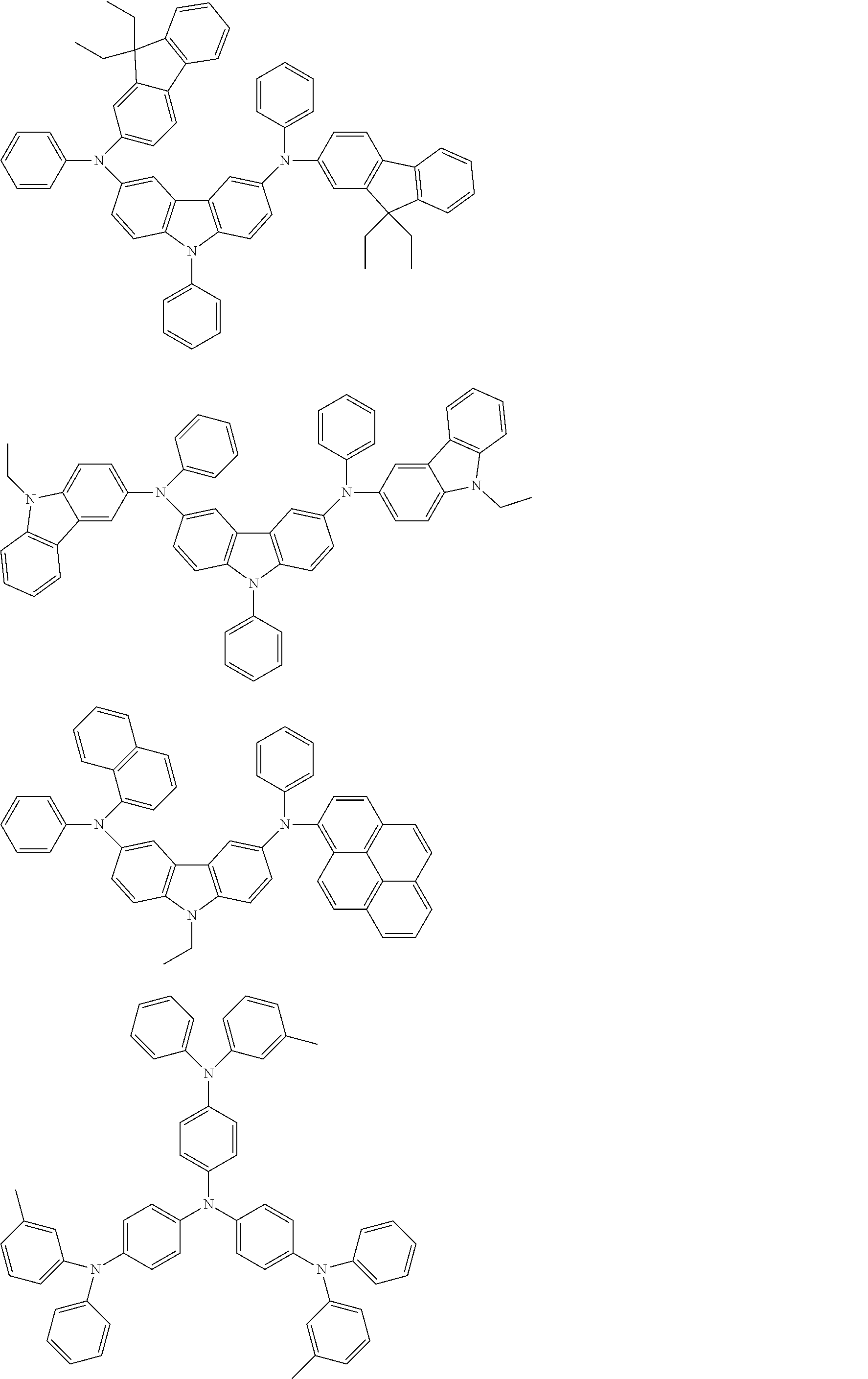

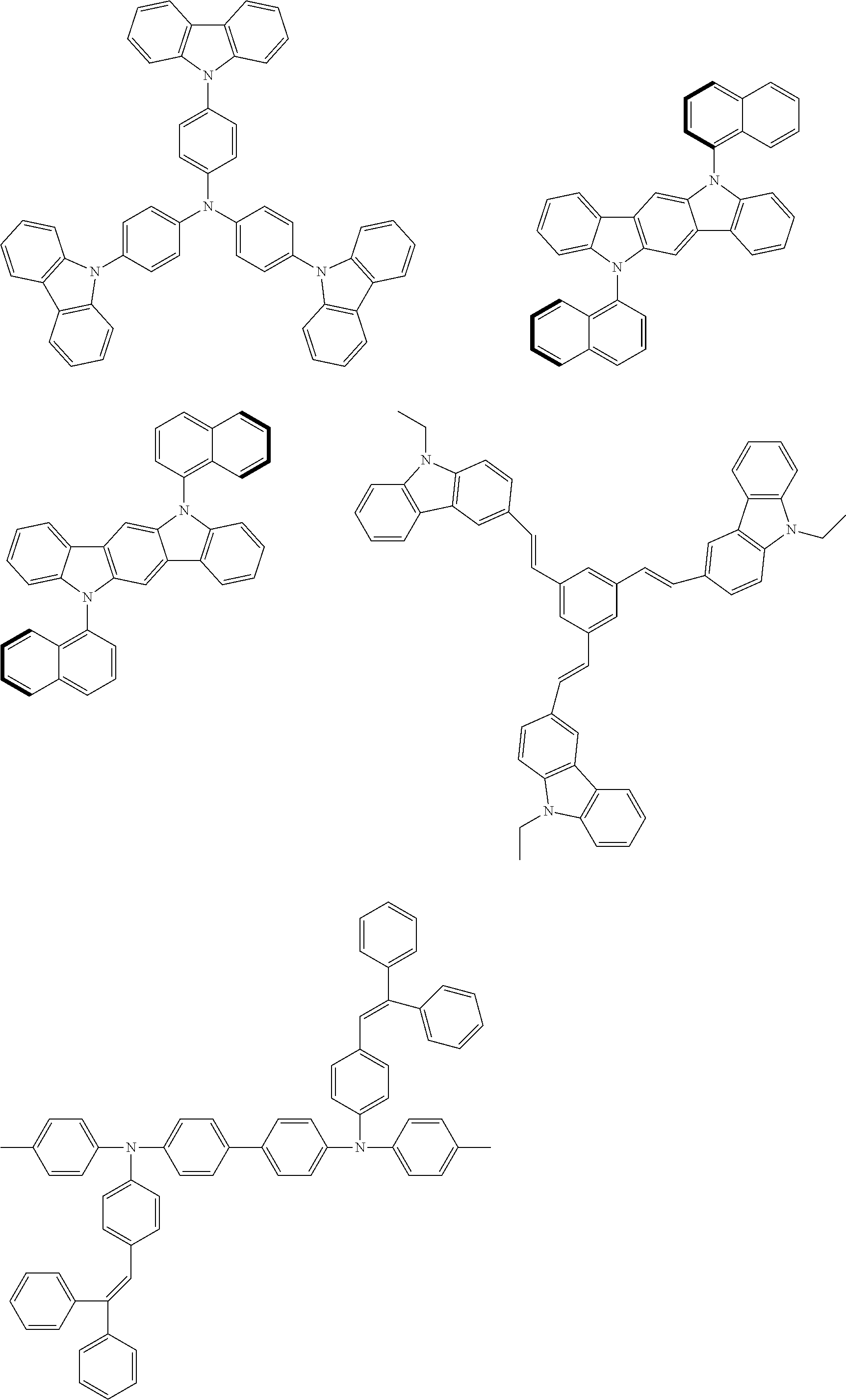

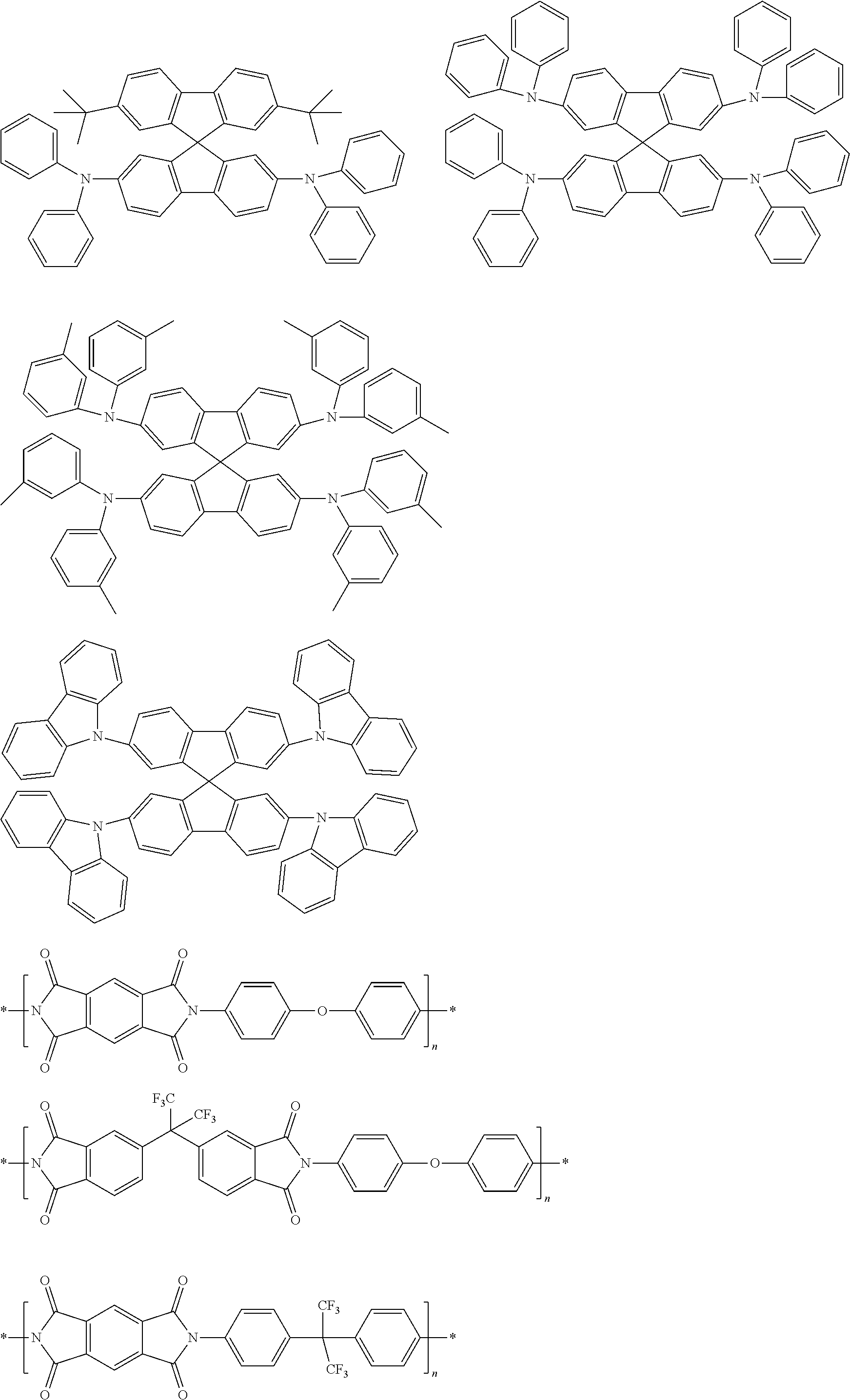

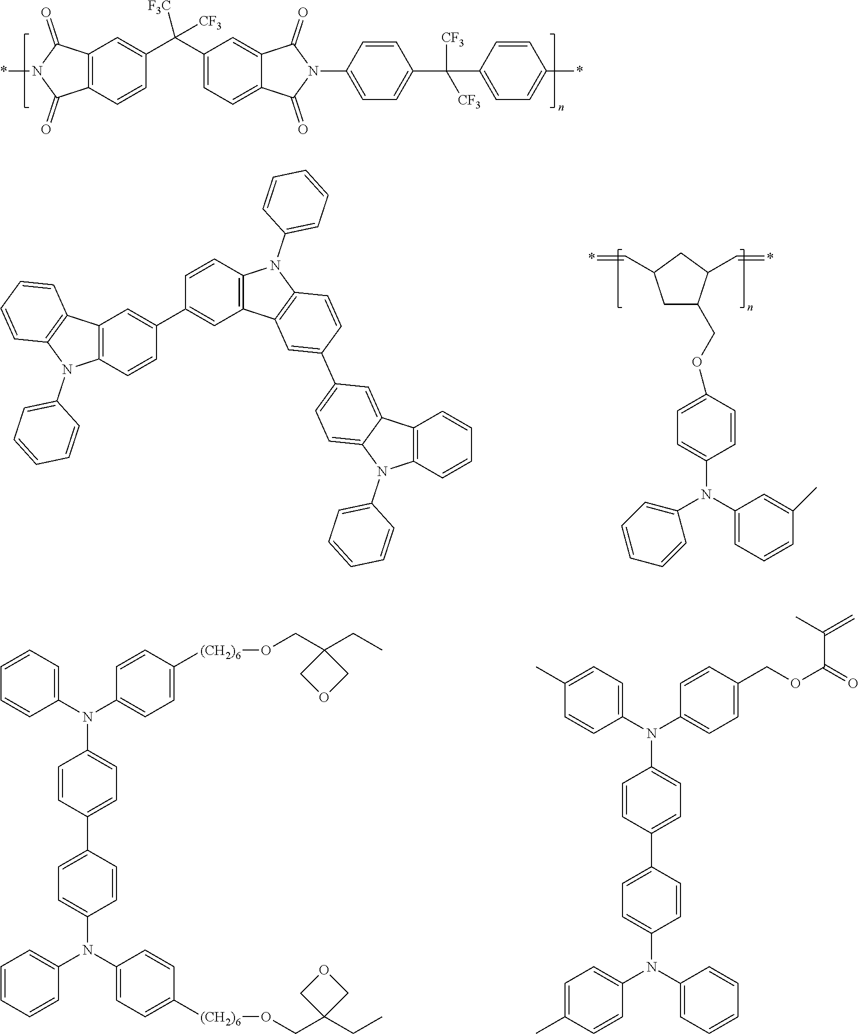

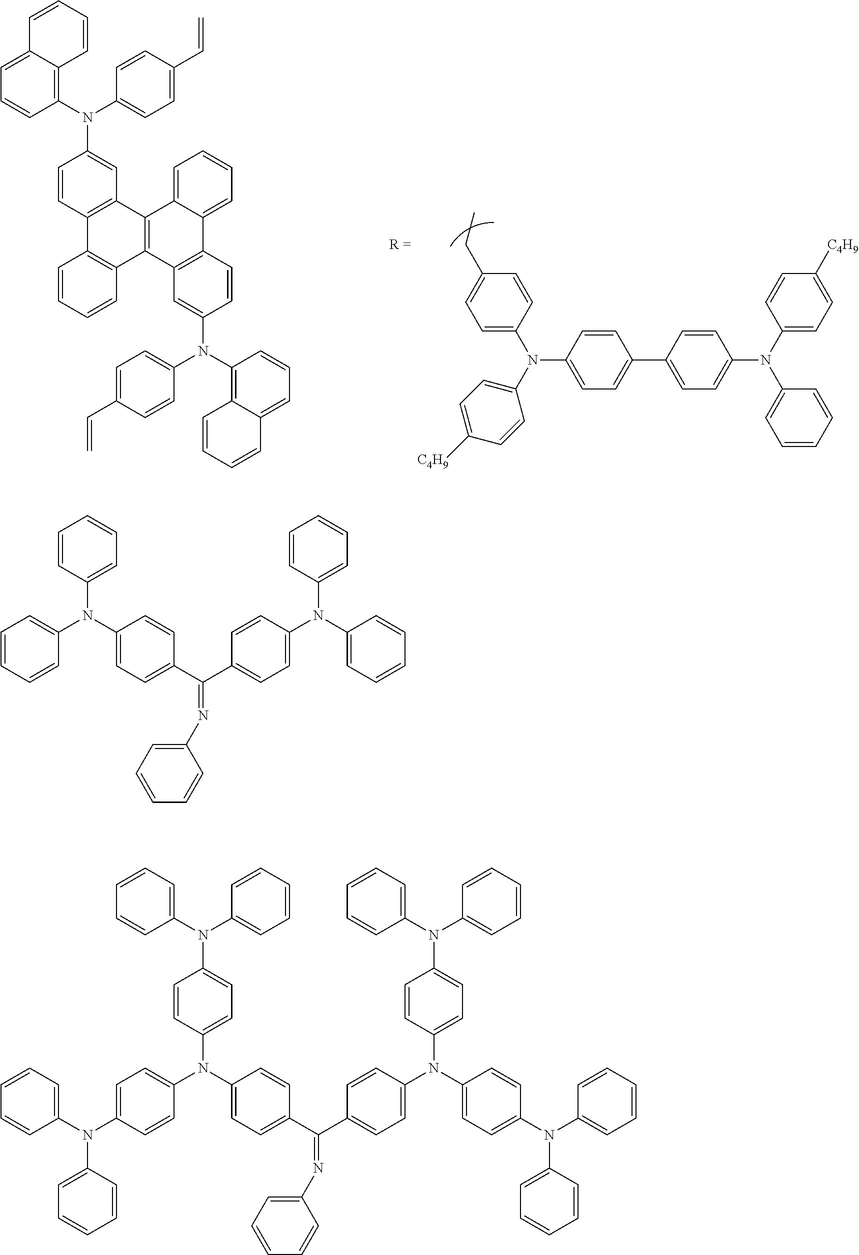

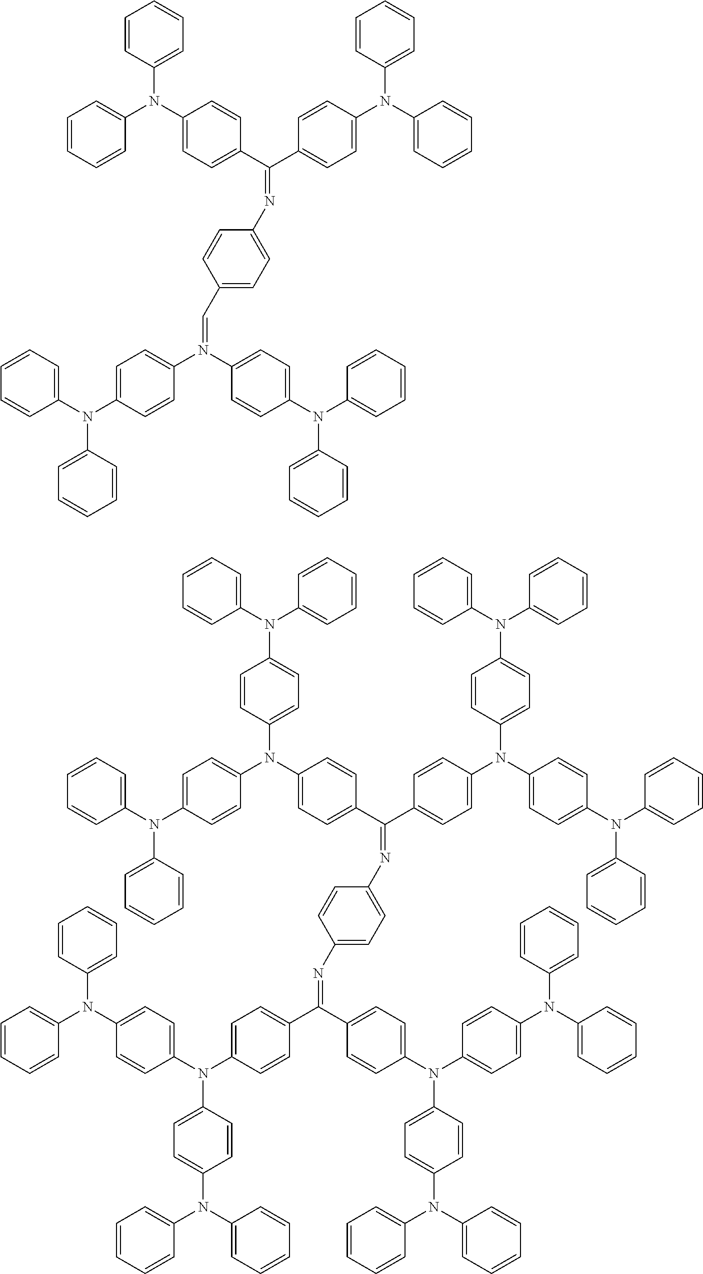

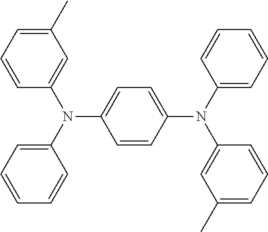 [SMILES: Cc1cccc(N(c2ccccc2)c2ccc(N(c3ccccc3)c3cccc(C)c3)cc2)c1]

Next, preferred examples of compounds usable as an electron blocking material are mentioned below.

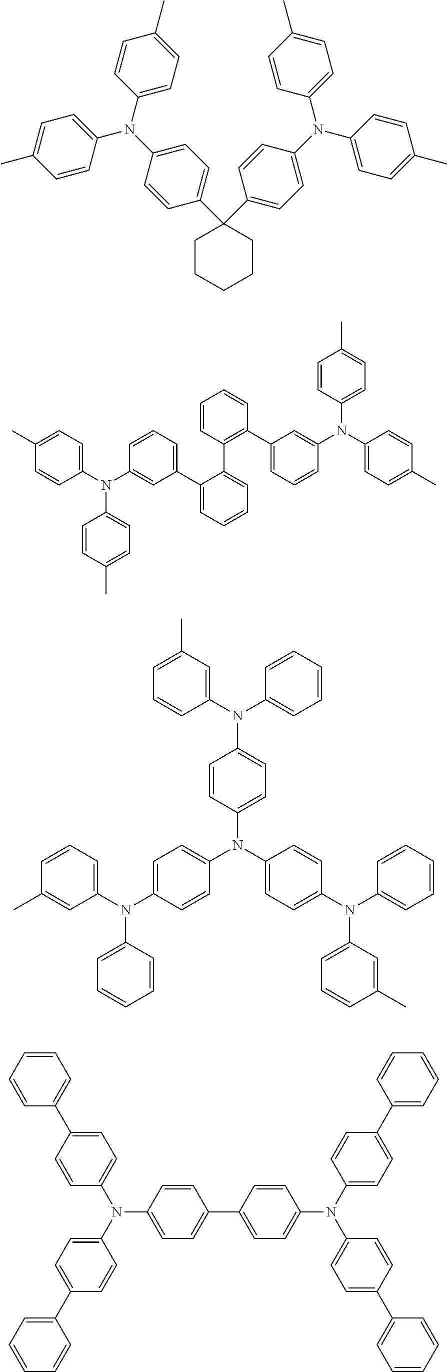

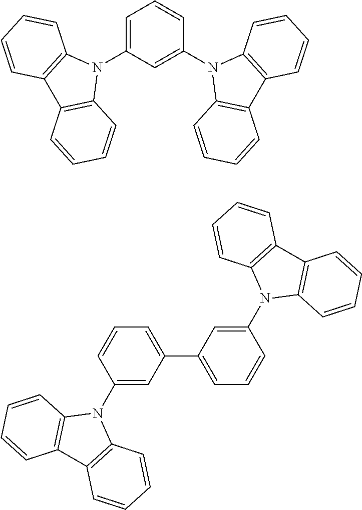

Next, preferred examples of compounds usable as ahole blocking material are mentioned below.

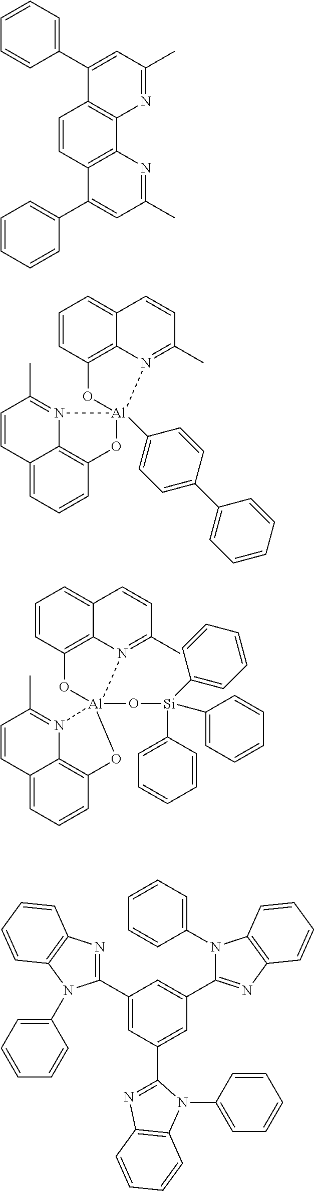

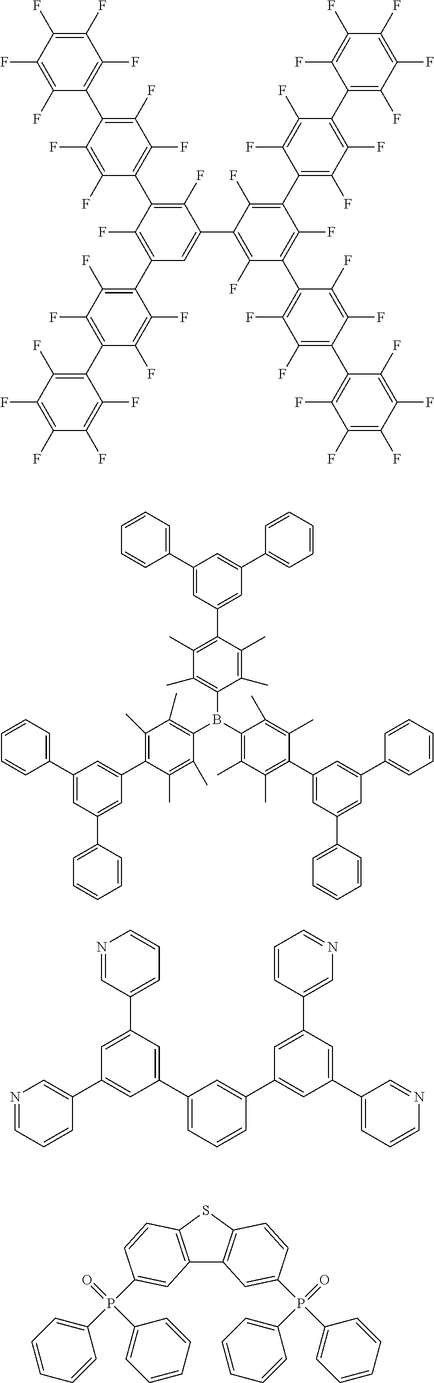

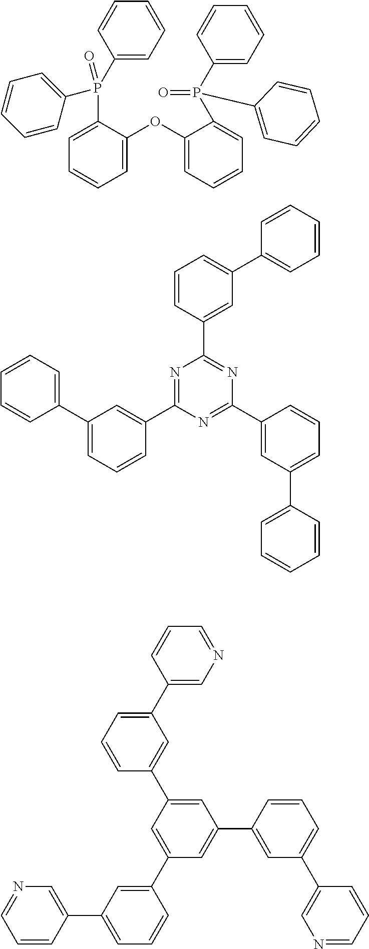 [SMILES: O=P(c1ccccc1)(c1ccccc1)c1ccccc1Oc1ccccc1P(=O)(c1ccccc1)c1ccccc1 ; c1ccc(-c2cccc(-c3nc(-c4cccc(-c5ccccc5)c4)nc(-c4cccc(-c5ccccc5)c4)n3)c2)cc1 ; c1cncc(-c2cccc(-c3cc(-c4cccc(-c5cccnc5)c4)cc(-c4cccc(-c5cccnc5)c4)c3)c2)c1]

Next, preferred examples of compounds usable as an electron transport material are mentioned below.

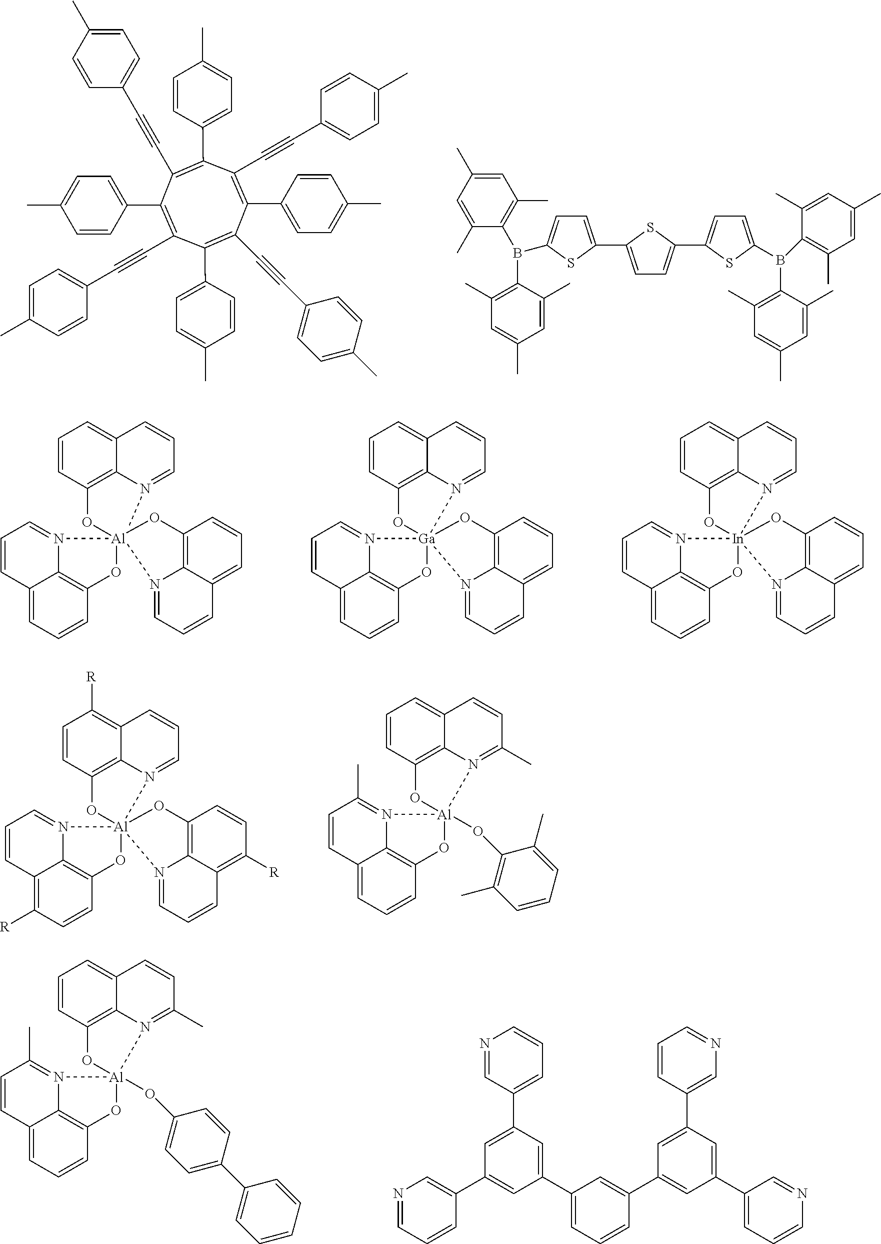

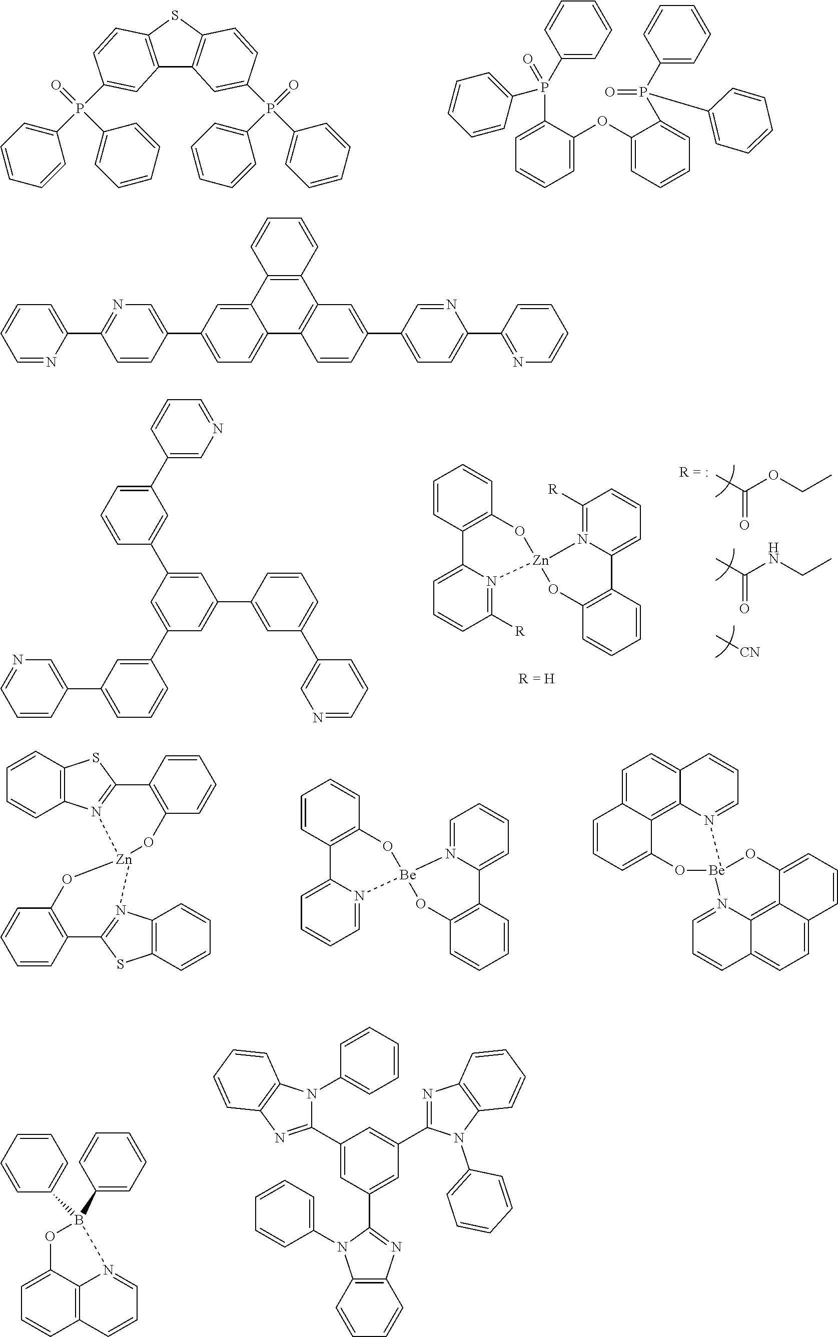

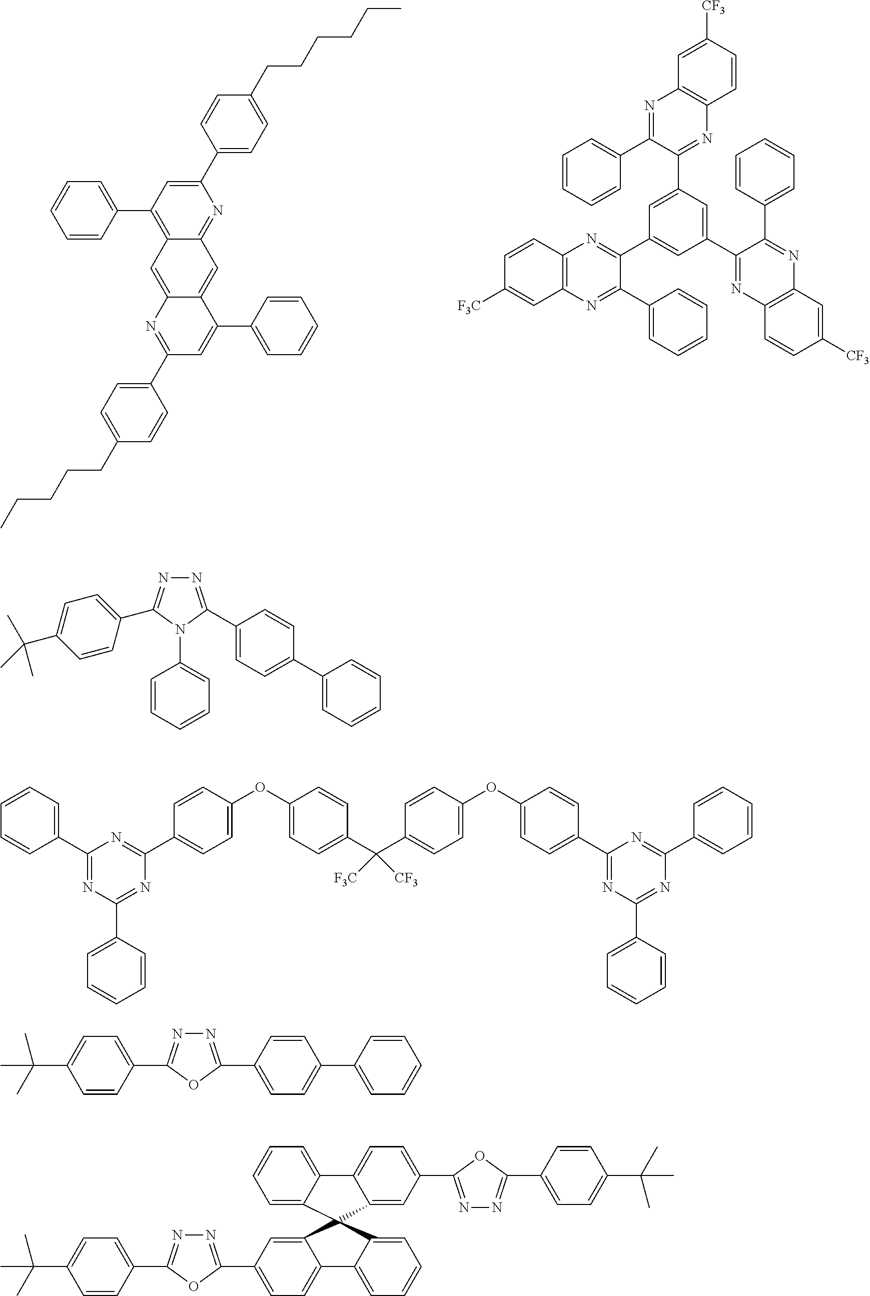

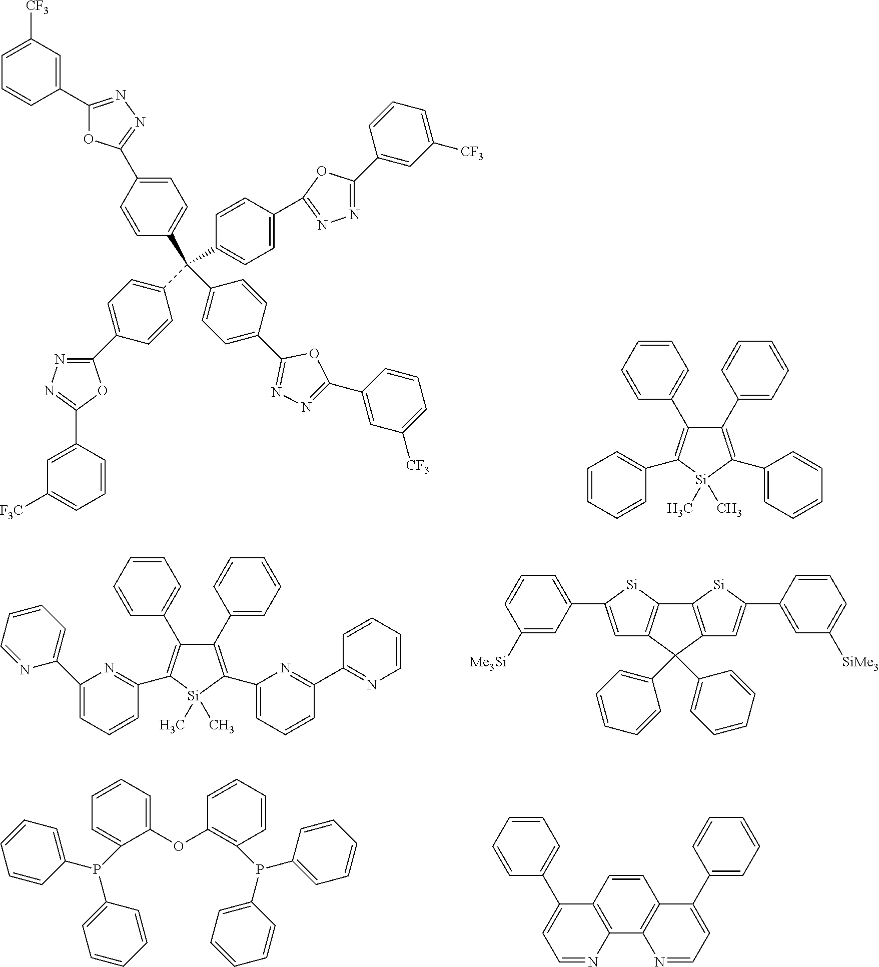

Next, preferred examples of compounds usable as an electron injection material are mentioned below.

LiF, CsF

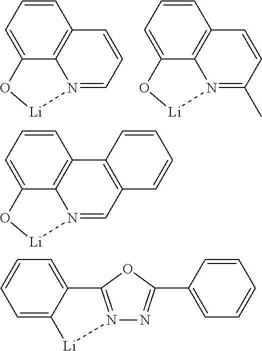

Further, preferred examples of compounds for use as additional materials are mentioned below. For example, these are considered to be added as a stabilization material.

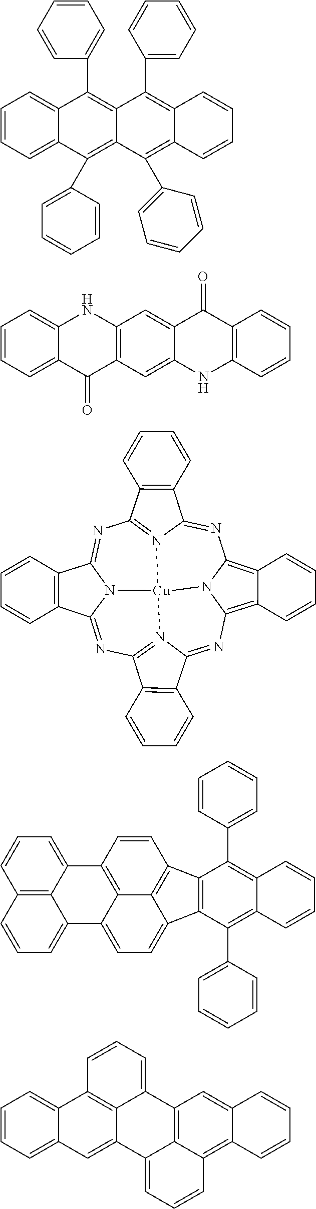

The organic electroluminescent device thus produced by the aforementioned method emits light on application of an electric field between the anode and the cathode of the device. In this case, when the light emission is caused by the excited singlet energy, light having a wavelength that corresponds to the energy level thereof may be confirmed as fluorescent light and delayed fluorescent light. When the light emission is caused by the excited triplet energy, light having a wavelength that corresponds to the energy level thereof may be confirmed as phosphorescent light. The normal fluorescent light has a shorter light emission lifetime than the delayed fluorescent light, and thus the light emission lifetime may be distinguished between the fluorescent light and the delayed fluorescent light.

On the other hand, the phosphorescent light may substantially not be observed a light-emitting material of an organic compound at room temperature since the excited triplet energy of the compound is unstable, and the rate constant of thermal deactivation thereof is large while the rate constant of light emission is small, and therefore the compound immediately deactivates. The excited triplet energy of an ordinary organic compound may be measured by observing light emission under an extremely low temperature condition.

The organic electroluminescent device of the invention may be applied to any of a single device, a structure with plural devices disposed in an array, and a structure having anodes and cathodes disposed in an X-Y matrix. According to the present invention using the compound represented by the formula (1) in a layer formed between an anode and a cathode, an organic light-emitting device markedly improved in point of at least one of drive voltage, light emission efficiency and device lifetime can be obtained. The organic light-emitting device such as the organic electroluminescent device of the present invention may be applied to a further wide range of purposes. For example, an organic electroluminescent display apparatus may be produced with the organic electroluminescent device of the invention, and for the details thereof, reference may be made to S. Tokito, C. Adachi and H. Murata, “Yuki EL Display” (Organic EL Display) (Ohmsha, Ltd.). In particular, the organic electroluminescent device of the invention may be applied to organic electroluminescent illumination and backlight which are highly demanded.

### EXAMPLES

The features of the present invention will be described more specifically with reference to Synthesis Examples and Examples given below. The materials, processes, procedures and the like shown below may be appropriately modified unless they deviate from the substance of the invention. Accordingly, the scope of the invention is not construed as being limited to the specific examples shown below. UV-visible light absorption spectra were measured using LAMBDA950-PKA (from Perkin Elmer Co., Ltd.); photoluminescence spectra were measured using Fluoromax-4 (from HORIBA Jobin Yvon GmbH); and device characteristics were evaluated using OLED IVL Characteristics Automatic IVL Measurement Device ETS-170 (from System Engineer's Co., Ltd.). In the present Examples, fluorescence having an emission lifetime of 0.05 μs or more was judged as delayed fluorescence.

### (Synthesis Example 1) Synthesis of Compound 1

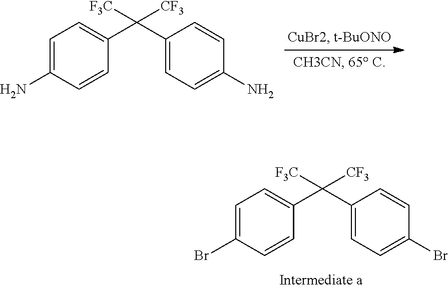 [SMILES: Nc1ccc(C(c2ccc(N)cc2)(C(F)(F)F)C(F)(F)F)cc1 ; FC(F)(F)C(c1ccc(Br)cc1)(c1ccc(Br)cc1)C(F)(F)F]

4,4′-(Perfluoropropane-2,2-diyl)dianiline (4.0 g, 12 mmol), tert-butyl nitrite (2.8 g, 27 mmol), copper(II) bromide (6.0 g, 27 mmol) and acetonitrile (30 mL) were put into a 100-mL flask, and heated at 65° C. for 2 hours. After cooled, 5% hydrochloric acid (30 mL) was added to the resultant mixture to stop the reaction, and extracted twice with dichloromethane (20 mL). The resultant organic layer was washed sequentially with water (5 mL), an aqueous 5% sodium hydrogencarbonate solution (30 mL) and saline water (30 mL). The organic layer was dried with anhydrous magnesium sulfate, filtered, and the filtrate was concentrated under reduced pressure. The resultant crude product was purified through silica gel column chromatography using a mixed solvent of chloroform/hexane=1/99 as an eluent, and concentrated and dried to give a white solid, 4,4′-(perfluoropropane-2,2-diyl)bis(bromobenzene) (intermediate a) at an actual yield of 4.8 g and a percent yield of 86%.

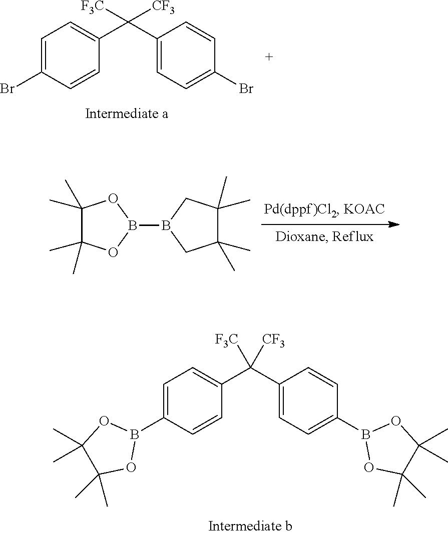

The intermediate a (4.6 g, 10.0 mmol), bis(pinacolato)diborane (7.6 g, 30.0 mmol), [1,1′-bis(diphenylphosphino)ferrocene]palladium(II) dichloride dichloromethane adduct (731 mg, 1.0 mmol) and potassium acetate (2.9 g, 30 mmol) were put into a 100-mL two-neck flask set with a magnetic stirrer, and dried for 10 minutes under reduced pressure. Dry dioxane (30 mL) was added to the mixture, stirred at room temperature for 30 minutes, and then heated at 100° C. for 24 hours. The reaction liquid was cooled down to room temperature, and then water was added to stop the reaction, and extracted with ethyl acetate. The resultant organic layer was dried with sodium sulfate, and concentrated under reduced pressure to give a crude product. The crude product was purified through silica gel column chromatography using a mixed solvent of chloroform/hexane=1/4 as an eluent, concentrated, dried, and then washed with hexane. The process gave a white solid, 2,2′-((perfluoropropane-2,2-diyl)bis(4,1-phenylene))bis(4,4,5,5-tetramethyl-1,3,2-dioxa bororane) (intermediate b) at an actual yield of 4.5 g and a percent yield of 80%.

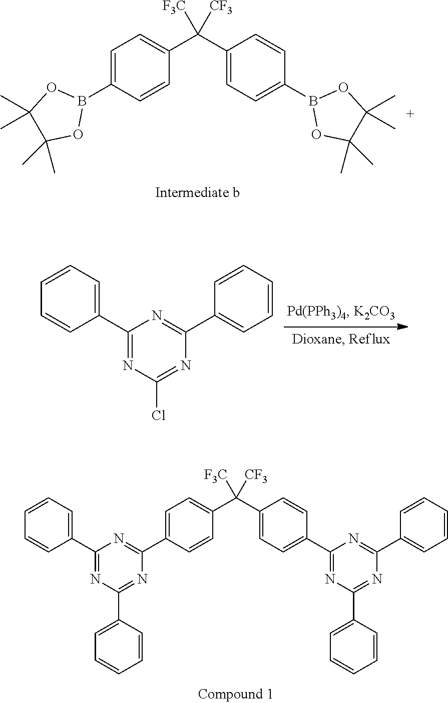

The intermediate b (2.78 g, 5.0 mmol), 2-chloro-4,6-diphenyl-1,3,5-triazine (1.34 g, 5.0 mmol), tetrakis(triphenylphosphine)palladium(0) (0.3 g, 0.27 mmol), potassium carbonate (1.38 g, 10.02 mmol), 1,4-dioxane (30 mL) and distilled water (10 mL) were put into a 100-mL round-bottomed flask, and refluxed in argon for 6 hours. The reaction liquid was extracted with chloroform, the resultant organic layer was dried with sodium sulfate, and concentrated with an evaporator to give a crude product. The crude product was purified through silica gel column chromatography using a mixed solvent of ethyl acetate/petroleum=1/4 as an eluent, then dried and concentrated to give a white solid, 6,6′((perfluoropropane-2,2-diyl)bis(4,1-phenylene))bis(2,4-diphenyl-1,3,5-triazine) (compound 1) at an actual yield of 2.36 g and a percent yield of 86%.

### (Synthesis Example 2) Synthesis of Compound 2

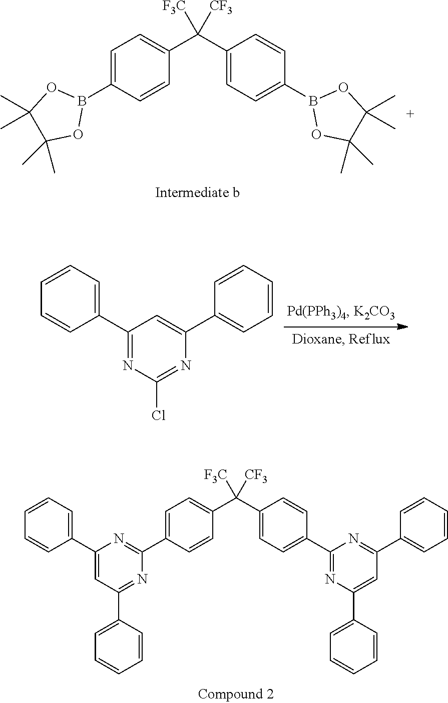

The intermediate b (2.78 g, 5.0 mmol), 2-chloro-4,6-diphenylpyrimidine (1.33 g, 5.0 mmol), tetrakis(triphenylphosphine)palladium(0) (0.3 g, 0.27 mmol), and potassium carbonate (1.38 g, 10.0 mmol) were put into a 100-mL round-bottomed flask, which was then purged with nitrogen. 1,4-Dioxane (30 mL) and distilled water (10 mL) were added to the mixture, and the mixture was stirred under reflux at 100° C. in a nitrogen atmosphere for 24 hours.

After stirred, the mixture was restored to room temperature, then led to pass through Celite to give a filtrate. The resultant filtrate was extracted with chloroform, and the extracted organic layer was concentrated with an evaporator to give a solid. The resultant solid was purified through silica gel column chromatography using a mixed solvent of hexane/ethyl acetate/chloroform=30/2/2. A fraction of the intended product was concentrated and dried to give a powdery white solid (compound 2).

### (Synthesis Example 3) Synthesis of Compound 4

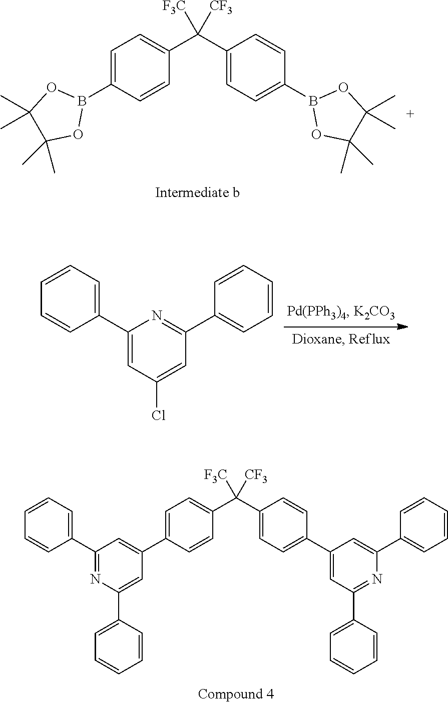

The intermediate b (2.78 g, 5.0 mmol), 4-chloro-2,6-diphenylpyridine (1.33 g, 5.0 mmol), tetrakis(triphenylphosphine)palladium(0) (0.3 g, 0.27 mmol), and potassium carbonate (1.38 g, 10.0 mmol) were put into a 100-mL round-bottomed flask, which was then purged with nitrogen. 1,4-Dioxane (30 mL) and distilled water (10 mL) were added to the mixture, and the mixture was stirred under reflux at 100° C. in a nitrogen atmosphere for 24 hours.

After stirred, the mixture was restored to room temperature, then led to pass through Celite to give a filtrate. The resultant filtrate was extracted with chloroform, and the extracted organic layer was concentrated with an evaporator to give a solid. The resultant solid was purified through silica gel column chromatography using a mixed solvent of hexane/ethyl acetate/chloroform=30/2/2. A fraction of the intended product was concentrated and dried to give a powdery white solid (compound 4).

### (Synthesis Example 4) Synthesis of Compound 23

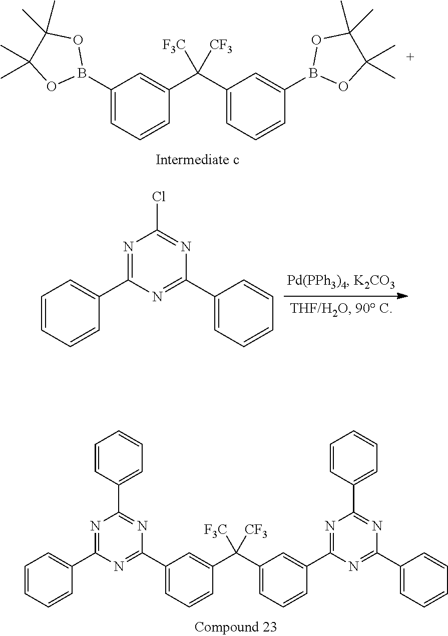

An intermediate c (3.63 g, 6.52 mmol) synthesized according to the same method as that for the intermediate b in Synthesis Example 1, 2-chloro-4,6-diphenyl-1,3,5-triazine (1.75 g, 6.52 mmol), tetrakis(triphenylphosphine)palladium(0) (0.381 g, 0.33 mmol) and potassium carbonate (2.70 g, 19.6 mmol) were put into a 100-mL round-bottomed flask, which was then purged with nitrogen. Tetrahydrofuran (30 mL) and distilled water (10 mL) were added to the mixture, and the mixture was stirred under reflux at 90° C. in a nitrogen atmosphere for 24 hours.

After stirred, the mixture was restored to room temperature, then led to pass through Celite to give a filtrate. The resultant filtrate was extracted with chloroform, and the extracted organic layer was concentrated with an evaporator to give a solid. The resultant solid was purified through silica gel column chromatography using a mixed solvent of hexane/ethyl acetate/chloroform=30/2/2. A fraction of the intended product was concentrated and dried to give a powdery white solid (compound 23) at an actual yield of 1.90 g and a percent yield of 38%.

¹H NMR (500 MHZ, CDC₃, δ): 9.05 (s, 2H), 8.87 (d, J=7.0 Hz, 2H), 8.70-8.73 (m, 8H), 7.56-7.68 (m, 16H); APCI-MS m/z: 766.30 M⁺.

### (Example 1) Preparation and Evaluation of Organic Photoluminescent Device Using Compound 1

A toluene solution of the compound 1 (concentration, 1×10−5 mol/L) was prepared in a glove box having an Ar atmosphere.

A UV-visible ray absorption spectrum, a photoluminescence spectrum at 298 K and a phosphorescence spectrum at 77 K of the toluene solution are shown in FIG. 2. In FIG. 2, “UV-Vis” indicates the UV-visible ray absorption spectrum, “PL” indicates the photoluminescence spectrum, and “Phos.” indicates the phosphorescence spectrum. The lowest excited triplet energy level of the compound 1, as derived from the phosphorescence spectrum thereof, was 3.0 eV.

### (Example 2) Production of Organic Electroluminescent Device Using Compound 1 as Host Material

On a glass substrate having, as formed thereon, an anode of indium tin oxide (ITO) having a thickness of 100 nm, thin films were laminated according to a vacuum evaporation method at a vacuum degree of 3×10⁻⁴ Pa. First, HAT-CN was formed on ITO in a thickness of 10 nm, and α-NPD was formed thereon in a thickness of 30 nm. Subsequently, Tris-PCz was formed in a thickness of 20 nm, and then mCBP was formed thereon in a thickness of 10 nm. Next, the compound 1 and 4CzIPN were co-evaporated from different evaporation sources to form a light-emitting layer having a thickness of 30 nm. At that time, the concentration of 4CzIPN was 15% by weight. On the formed light-emitting layer, the compound 1 was layered in a thickness of 10 nm, and Bebq₂ was layered thereon in a thickness of 35 nm. Further, lithium fluoride (LiF) was vapor-deposited in a thickness of 0.8 nm, and then aluminum (Al) was vapor-deposited in a thickness of 100 nm to form a cathode, thereby producing an organic electroluminescent device.

### (Comparative Example 1) Production of Organic Electroluminescent Device Using mCBP

An organic electroluminescent device was produced in the same manner as in Example 1, except that mCBP was used in place of the compound 1 in forming the light-emitting layer, and a layer of T2T having a thickness of 10 nm was formed in place of forming a layer of the compound 1 on the light-emitting layer.

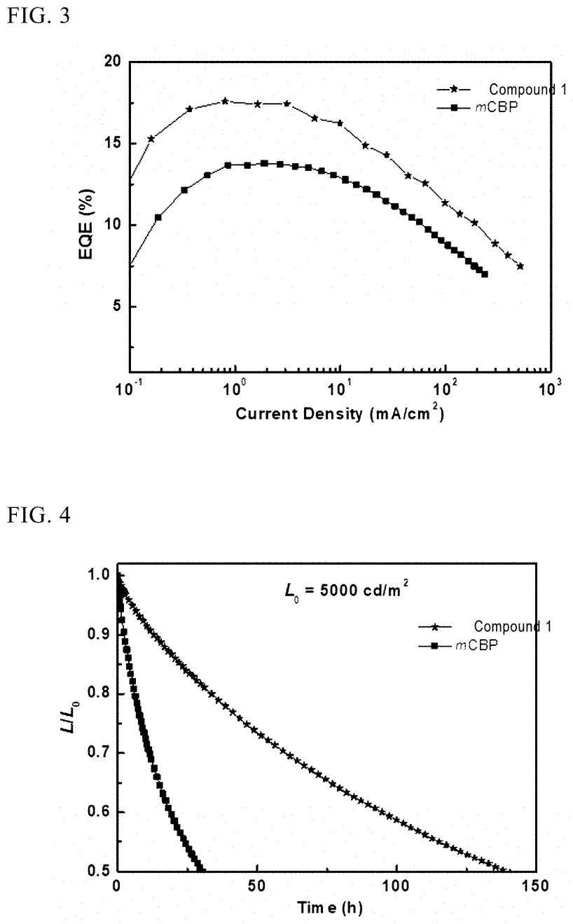

The organic electroluminescent devices produced in Example 1 and Comparative Example 1 were analyzed to measure the external quantum efficiency (EQE)-current density characteristic thereof, and the results are shown in FIG. 3. The results of measurement of the time-dependent change of the luminance ratio L/L₀ thereof are shown in FIG. 4. The luminance ratio L/L₀ plotted on the vertical axis in FIG. 4 is a value of a ratio of the luminance L at an elapsed time to the initial luminance L₀, and the initial luminance L₀ is 5000 cd/m². In FIGS. 3 and 4, “Compound 1” indicates the organic electroluminescent device of Example 1 using the compound 1 as a host material, and “mCBP” indicates the organic electroluminescent device of Comparative Example 1 using mCBP as a host material.

From FIGS. 3 and 4, it is known that the organic electroluminescent device of Example 1 using the compound 1 as a host material has a dramatically higher external quantum efficiency and has a far longer lifetime, as compared with the organic electroluminescent device of Comparative Example 1 using mCBP as a host material.

### (Example 3) Production of Organic Electroluminescent Device Using Compound 1 as Hole Blocking Material and Electron Transport Material

On a glass substrate having, as formed thereon, an anode of indium tin oxide (ITO) having a thickness of 100 nm, thin films were laminated according to a vacuum evaporation method at a vacuum degree of 3×10⁻⁴ Pa. First, HAT-CN was formed on ITO in a thickness of 10 nm, and α-NPD was formed thereon in a thickness of 10 nm. Subsequently, Tris-PCz was formed in a thickness of 15 nm, and then mCBP was formed thereon in a thickness of 5 nm. Next, mCBP and 4CzIPN were co-evaporated from different evaporation sources to form a light-emitting layer having a thickness of nm. At that time, the concentration of 4CzIPN was 20% by weight. On the formed light-emitting layer, the compound 1 was layered in a thickness of 10 nm, and a co-deposited layer of the compound 1 and Liq was formed thereon in a thickness of 40 nm. At that time, the concentration of Liq was 30% by weight. Further, Liq was vapor-deposited in a thickness of 2 nm, and then aluminum (Al) was vapor-deposited in a thickness of 100 nm to form a cathode, thereby producing an organic electroluminescentdevice.

### (Comparative Example 2) Production of Organic Electroluminescent Device Using SF3-TRZ

An organic electroluminescent device was produced in the same manner as in Example 2, except that SF3-TRZ was used in place of the compound 1 in forming the electron transport layer.

The organic electroluminescent devices produced in Example 2 and Comparative Example 2 were compared in point of the voltage value at 100 mA/cm², the maximum external quantum efficiency EQE and the time LT80 taken until the luminance ratio L/L₀ reached 0.8. The initial luminance L₀ is 5000 cd/m².

The maximum external quantum efficiency EQE in both Example 2 and Comparative Example 2 reached 20%. The voltage value in Example 2 was lower by about 2 V than that in Comparative Example 2, that is, Example 2 attained drive voltage reduction by 2 V, and LT80 in Example 2 increased by 2.95 times.

From these results, it is known that the organic electroluminescent device of Example 2 using the compound 1 as a hole blocking material and an electron transport material needs a lower drive voltage and has a far longer device lifetime, as compared with the electroluminescent device of Comparative Example 2 using SF3-TRZ as a hole blocking material and an electron transport material.

### (Example 4) Production of Organic Electroluminescent Device Using Compound 1 as Hole Blocking Material and Electron Transport Material

On a glass substrate having, as formed thereon, an anode of indium tin oxide (ITO) having a thickness of 100 nm, thin films were laminated according to a vacuum evaporation method at a vacuum degree of 3×10⁻⁴ Pa. First, HAT-CN was formed on ITO in a thickness of 10 nm, and α-NPD was formed thereon in a thickness of 10 nm. Subsequently, Tris-PCz was formed in a thickness of 15 nm, and then mCBP was formed thereon in a thickness of 5 nm. Next, H-1 and 4CzIPN were co-evaporated from different evaporation sources to form a light-emitting layer having a thickness of nm. At that time, the concentration of 4CzIPN was 20% by weight. On the formed light-emitting layer, the compound 1 was layered in a thickness of 50 nm, and Liq was formed thereon in a thickness of 2 nm, and then aluminum (Al) was vapor-deposited in a thickness of 100 nm to form a cathode, thereby producing an organic electroluminescent device.

### (Comparative Example 3) Production of Organic Electroluminescent Device Using SF3-TRZ

An organic electroluminescent device was produced in the same manner as in Example 4, except that SF3-TRZ was formed in a thickness of 10 nm as a hole blocking layer in place of forming the layer of the compound 1 in a thickness of 50 nm, and SF3-TRZ and Liq were co-deposited thereon in a co-evaporation ratio of 7/3 as an electron transport layer in a thickness of 40 nm.

The organic electroluminescent devices produced in Example 4 and Comparative Example 3 were driven to measure the drive voltage thereof at 100 mA/cm², and the drive voltage was 7.9 V in Example 4 and was 8.9 V in Comparative Example 3. The time L95 taken until the luminance ratio L/L₀ at 5000 cm/m² could reach 0.95 was measured, and the time in Example 4 was 1.8 hours, and was 1.0 hour in Comparative Example 3. In that way, the device in Example 4 achieved drive voltage reduction by 1 V as compared with that in Comparative Example 3, and LT80 of the former was 1.8 times the latter.

From these results, it is known that the compound 1 is useful as a hole blocking material and an electron transport material.

### (Example 5) Production of Blue-Emitting Organic Electroluminescent Device Using Compound 1 as Hole Blocking Material

On a glass substrate having, as formed thereon, an anode of indium tin oxide (ITO) having a thickness of 50 nm, thin films were laminated according to a vacuum evaporation method at a vacuum degree of 3×10⁻⁴ Pa. First, HAT-CN was formed on ITO in a thickness of 10 nm, and α-NPD was formed thereon in a thickness of 15 nm. Subsequently, Tris-PCz was formed in a thickness of 15 nm, and then PYD-2Cz was formed thereon in a thickness of 5 nm. Next, PYD-2Cz and D-1 were co-evaporated from different evaporation sources to form a light-emitting layer having a thickness of nm. At that time, the concentration of PYD-2Cz was 30% by weight. On the formed light-emitting layer, the compound 1 was layered in a thickness of 10 nm, and a co-deposited film of SF3-TRZ and Liq was layered thereon in a thickness of 30 nm. At that time, the concentration of Liq was 30% by weight. Further, Liq was vapor-deposited in a thickness of 2 nm, and then aluminum (Al) was vapor-deposited in a thickness of 100 nm to form a cathode, thereby producing an organic electroluminescent device.

### (Example 6) Production of Blue-Emitting Organic Electroluminescent Device Using Compound 23 as Hole Blocking Material

An organic electroluminescent device was produced in the same manner as in Example 5, except that the compound 23 was used in place of the compound 1 in forming the hole blocking layer.

### (Comparative Example 4) Production of Organic Electroluminescent Device Using SF3-TRZ

An organic electroluminescent device was produced in the same manner as in Example 5, except that SF3-TRZ was used in place of the compound 1 in forming the hole blocking layer.

Regarding the organic electroluminescent devices produced in Example 5, Example 6 and Comparative Example 4, EQE at 1000 cd/m² of each device was measured, and was 16.0% in Example 5, 17.3% in Example 6, and 13.0% in Comparative Example 4. In that manner, Example 5 achieved 3.0% EQE increase over Comparative Example 4, and Example 6 achieved 4.3% EQE increase over Comparative Example 4.

From these results, it is known that the lowest excited triplet energy level (E_{T1}) of the compound 1 and the compound 23 is high, and the compounds are useful for blue-emitting organic electroluminescent devices.

E_{T1} of the compounds 1 to 7, 12, 14, 17, 19 and 21 to 23 was calculated according to a computational chemical technique. In the computational chemical technique, Q-Chem 5.1 Program from Q-Chem, Inc. was used. Here, for optimization of the molecular structure and calculation of the electron condition in a ground singlet state S₀, a B3LYP/6-31G(d) method was employed, and for calculation of the lowest excited triplet energy level (E_{T1}), a time-dependent density-functional theory (TD-DFT) method was employed. The results are shown in the following Table. A measured value (solution) of E_{T1} of the compound 1 was 3.00 eV, a calculated value thereof was 3.02 eV; a measured value (solution) of E_{T1} of the compound 23 was 3.04 eV, a calculated value thereof was 3.03 eV. From this, it is confirmed that the calculated value and the measured value are extremely close to each other, and it is verified that the calculation accuracy is high. From the calculation results of the other compounds in Table 1, it is known that the other compounds also have a high E_{T1} and are useful for blue-emitting organic electroluminescent devices.

TABLE 1

| Compound | E_{T1} (eV) |
|---|---|
| SF3-TRZ | 2.86 |
| Compound 1 | 3.02 |
| Compound 23 | 3.03 |
| Compound 2 | 2.97 |
| Compound 3 | 2.97 |
| Compound 4 | 2.99 |
| Compound 5 | 2.98 |
| Compound 6 | 3.06 |
| Compound 7 | 3.06 |
| Compound 12 | 2.97 |
| Compound 14 | 2.97 |
| Compound 17 | 3.01 |
| Compound 19 | 3.01 |
| Compound 21 | 2.99 |
| Compound 22 | 3.06 |

### (Example 7) Production of Light-Emitting Organic Electroluminescent Device Using Compound 1 as Hole Blocking Material and Electron Transport Material

On a glass substrate having, as formed thereon, an anode of indium tin oxide (ITO) having a thickness of 100 nm, thin films were laminated according to a vacuum evaporation method at a vacuum degree of 3×10⁻⁴ Pa. First, HAT-CN was formed on ITO in a thickness of 10 nm, and α-NPD was formed thereon in a thickness of 10 nm. Subsequently, Tris-PCz was formed in a thickness of 15 nm, and then mCBP was formed thereon in a thickness of 5 nm. Next, H-1 and 4CzIPN were co-evaporated from different evaporation sources to form a light-emitting layer having a thickness of nm. At that time, the concentration of 4CzIPN was 20% by weight. On the formed light-emitting layer, the compound 1 was layered in a thickness of 10 nm, and a co-deposited layer of the compound 1 and Liq was formed thereon in a thickness of 40 nm. At that time, the concentration of Liq was 30% by weight. Further, Liq was vapor-deposited in a thickness of 2 nm, and then aluminum (Al) was vapor-deposited in a thickness of 100 nm to form a cathode, thereby producing an organic electroluminescentdevice.

### (Comparative Example 5) Production of Organic Electroluminescent Device Using SF3-TRZ

An organic electroluminescent device was produced in the same manner as in Example 7, except that SF3-TRZ was used in place of the compound 1 in forming the hole blocking layer and the electron transport layer.

Regarding the organic electroluminescent devices produced in Example 7 and Comparative Example 5, the drive voltage at 5000 cd/m² of each device was measured, and was 5.6 V in Example 7 and 6.3 V in Comparative Example 5. The time LT95 taken until the luminance ratio L/L₀ reached 0.95 was measured, and was about 140 hours in Example 7 and 66 hours in Comparative Example 5. From these results, it is known that the compound 1 is useful as a hole blocking material and an electron transport material.

### (Example 8) Production of Light-Emitting Organic Electroluminescent Device Using Compound 1 as Hole Blocking Material

An organic electroluminescent device was produced in the same manner as in Example 7, except that a co-deposited film of SF3-TRZ and Liq was formed in place of forming the co-deposited film of the compound 1 and Liq as the electron transport layer.

Regarding the organic electroluminescent devices produced in Example 8 and Comparative Example 5, EQE at 10000 cd/m² of each device was measured, and was 11.8% in Example 8 and 10.4% in Comparative Example 5. From the results, it is known that the compound 1 is useful in point of increasing light emission efficiency.

### (Example 9) Production of Light-Emitting Organic Electroluminescent Device Using Compound 1 as Hole Blocking Material

On a glass substrate having, as formed thereon, an anode of indium tin oxide (ITO) having a thickness of 50 nm, thin films were laminated according to a vacuum evaporation method at a vacuum degree of 3×10⁻⁴ Pa. First, HAT-CN was formed on ITO in a thickness of 10 nm, and α-NPD was formed thereon in a thickness of 15 nm. Subsequently, Tris-PCz was formed in a thickness of 15 nm, and then PYD-2Cz was formed thereon in a thickness of 5 nm. Next, PYD-2Cz and D-1 were co-evaporated from different evaporation sources to form a light-emitting layer having a thickness of 30 nm. At that time, the concentration of D-1 was 30% by weight. On the formed light-emitting layer, the compound 1 was layered in a thickness of 10 nm, and a co-deposited layer of SF3-TRZ and Liq was formed thereon in a thickness of 30 nm. At that time, the concentration of Liq was 30% by weight. Further, Liq was vapor-deposited in a thickness of 2 nm, and then aluminum (Al) was vapor-deposited in a thickness of 100 nm to form a cathode, thereby producing an organic electroluminescentdevice.

### (Example 10) Production of Light-Emitting Organic Electroluminescent Device Using Compound 1 as Hole Blocking Layer

An organic electroluminescent device was produced in the same manner as in Example 9, except that a co-deposited film of TRZ-4DPBT and Liq was formed in place of forming the co-deposited film of SF3-TRZ and Liq as the electron transport layer.

### (Comparative Example 6) Production of Organic Electroluminescent Device Using SF3-TRZ

An organic electroluminescent device was produced in the same manner as in Example 9, except that SF3-TRZ was used in place of the compound 1 in forming the hole blocking layer.

Regarding the organic electroluminescent devices produced in Example 9, Example 10 and Comparative Example 6, EQE at 10000 cd/m² of each device was measured, and was 16.0% in Example 9, 16.6% in Example 10 and 13.7% in Comparative Example 6. From the results, it is known that the compound 1 is useful in point of increasing light emission efficiency.

### (Example 11) Production of Light-Emitting Organic Electroluminescent Device Using Compound 23 as Hole Blocking Material

An organic electroluminescent device was produced in the same manner as in Example 9, except that the compound 23 was used in place of the compound 1 in forming the hole blocking layer.

### (Example 12) Production of Light-Emitting Organic Electroluminescent Device Using Compound 23 as Hole Blocking Material and Electron Transport Material

An organic electroluminescent device was produced in the same manner as in Example 11, except that a co-deposited film of the compound 23 and Liq was formed in place of forming the co-deposited film of SF3-TRZ and Liq as the electron transport layer.

Regarding the organic electroluminescent devices produced in Example 11, Example 12 and Comparative Example 6, EQE at 10000 cd/m² of each device was measured, and was 17.3% in Example 11, 14.0%/in Example 12 and 13.0% in Comparative Example 6. From the results, it is known that the compound 23 is useful in point of increasing light emission efficiency.

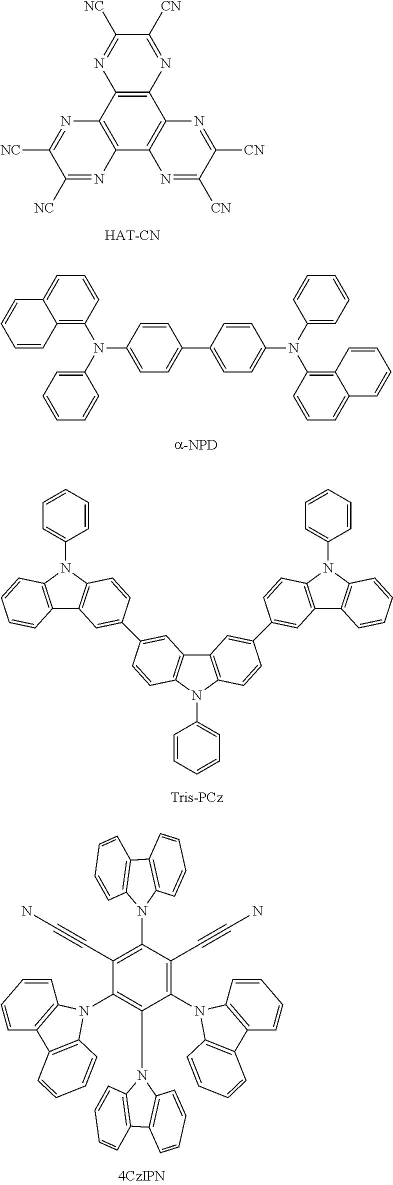 [SMILES: N#Cc1nc2c3nc(C#N)c(C#N)nc3c3nc(C#N)c(C#N)nc3c2nc1C#N ; c1ccc(N(c2ccc(-c3ccc(N(c4ccccc4)c4cccc5ccccc45)cc3)cc2)c2cccc3ccccc23)cc1 ; c1ccc(-n2c3ccccc3c3cc(-c4ccc5c(c4)c4cc(-c6ccc7c(c6)c6ccccc6n7-c6ccccc6)ccc4n5-c4ccccc4)ccc32)cc1 ; NC#Cc1c(-n2c3ccccc3c3ccccc32)c(C#CN)c(-n2c3ccccc3c3ccccc32)c(-n2c3ccccc3c3ccccc32)c1-n1c2ccccc2c2ccccc21]

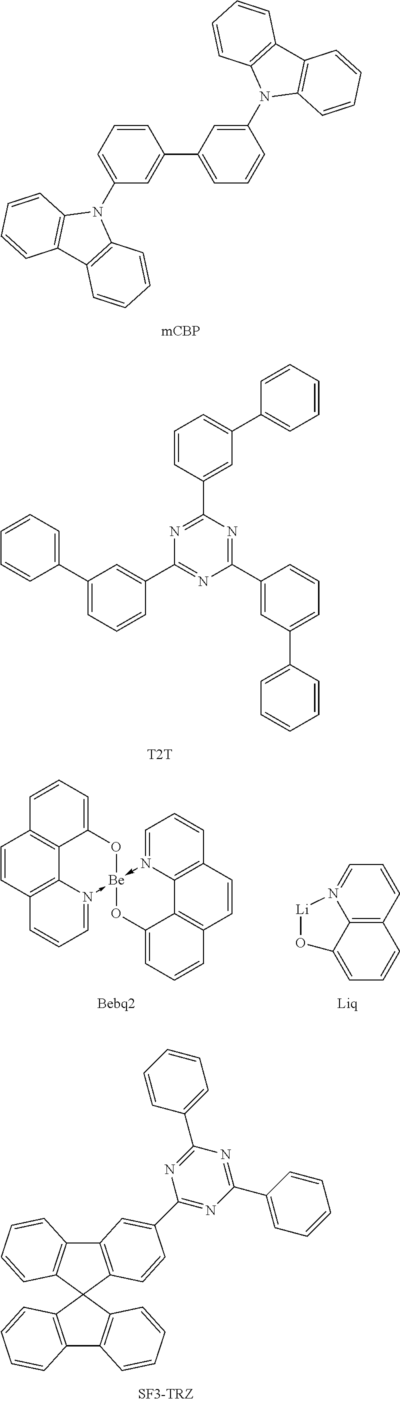

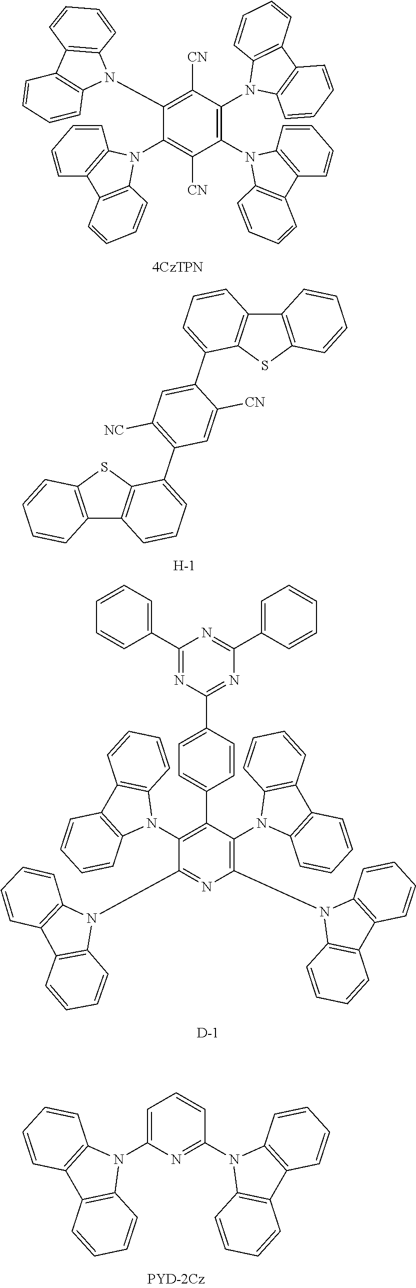

### INDUSTRIAL APPLICABILITY

The compound of the present invention is useful as a charge transport material. Therefore, the compound of the present invention is effectively used as a charge transport material for organic light-emitting devices such as organic electroluminescent devices, and consequently, it becomes possible to provide an organic light-emitting device that has realized at least one of a low drive voltage, a high light emission efficiency, and a long device lifetime. Accordingly, the industrial applicability of the present invention is high.

### REFERENCE SIGNS LIST

1 Substrate
2 Anode
3 Hole Injection Layer
4 Hole Transport Layer
5 Light-Emitting Layer
6 Electron Transport Layer
7 Cathode

1-21. (canceled)

22. A compound represented by the following general formula (1): 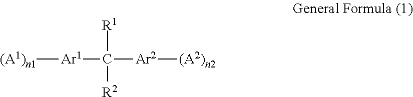
wherein R¹ and R² each independently represent a fluoroalkyl group,
Ar¹ and Ar² each independently represent an aromatic ring optionally having a substituent,
A¹ and A² each independently represent an aryl group substituted with a group having a positive Hammett σp value, or an aryl group substituted with a phenyl group, or represents a substituted or unsubstituted heteroaryl group bonding to Ar¹ or Ar² via a carbon atom (but excluding a substituted or unsubstituted imidazolyl group bonding to Ar¹ or Ar² via a carbon atom, a substituted or unsubstituted thiadiazolyl group bonding to Ar¹ or Ar² via a carbon atom, and a substituted or unsubstituted oxadiazolyl group bonding to Ar¹ or Ar² via a carbon atom),
n1 represents a natural number not more than the maximum number of substituents substitutable on Ar¹,
n2 represents a natural number not more than the maximum number of substituents substitutable on Ar².

23. An organic light-emitting device having a layer containing a compound represented by the following general formula (1), on a substrate: 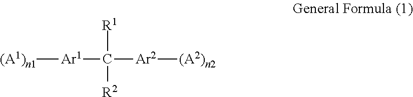
wherein R¹ and R² each independently represent a fluoroalkyl group,
Ar¹ and Ar² each independently represent an aromatic ring optionally having a substituent,
A¹ and A² each independently represent an aryl group substituted with a group having a positive Hammett σp value, or an aryl group substituted with a phenyl group, or represents a substituted or unsubstituted heteroaryl group bonding to Ar¹ or Ar² via a carbon atom (but excluding a substituted or unsubstituted imidazolyl group bonding to Ar¹ or Ar² via a carbon atom, a substituted or unsubstituted thiadiazolyl group bonding to Ar¹ or Ar² via a carbon atom, and a substituted or unsubstituted oxadiazolyl group bonding to Ar¹ or Ar² via a carbon atom),
n1 represents a natural number not more than the maximum number of substituents substitutable on Ar¹,
n2 represents a natural number not more than the maximum number of substituents substitutable on Ar².

24. The organic light-emitting device according to claim 23,
containing a compound such that a difference ΔE_{ST} between the lowest excited singlet energy level and the lowest excited triplet energy level thereof is 0.3 eV or less, in the light-emitting layer.

25. The organic light-emitting device according to claim 23, which emits delayed fluorescence.

26. (canceled)

27. The organic light-emitting device according to claim 23, having a light-emitting layer, wherein the compound represented by the general formula (1) is contained in the light-emitting layer in an amount of 50% by weight or more.

28. The organic light-emitting device according to claim 23, having a light-emitting layer, anode and cathode, wherein the compound represented by the general formula (1) is contained in a layer formed between the light-emitting layer and the cathode.

29. The organic light-emitting device according to claim 23, wherein R¹ and R² each are independently a perfluoroalkyl group.

30. The organic light-emitting device according to claim 23, wherein R¹ and R² each are a trifluoromethyl group.

31. The organic light-emitting device according to claim 23, wherein Ar¹ and Ar² each are a benzene ring unsubstituted at the bonding positions other than the bonding positions to A¹ or A² and the bonding position to C to which R¹ and R² bond.

32. The organic light-emitting device according to claim 23, wherein the substituted or unsubstituted heteroaryl group is a group containing at least one or more of a pyridine ring, a pyrimidine ring and a triazine ring.

33. The organic light-emitting device according to claim 23, wherein the substituted or unsubstituted heteroaryl group is a substituted or unsubstituted pyridinyl group, a substituted or unsubstituted pyrimidinyl group, or a substituted or unsubstituted triazinyl group.

34. The organic light-emitting device according to claim 23, wherein the substituted or unsubstituted heteroaryl group is a group containing a triazine ring.

35. The organic light-emitting device according to claim 23, wherein the substituted or unsubstituted heteroaryl group is a substituted or unsubstituted triazinyl group.

36. The organic light-emitting device according to claim 23, wherein the heteroaryl group is a heteroaryl group substituted with a substituted or unsubstituted aryl group.

37. The organic light-emitting device according to claim 23, wherein the heteroaryl group is a triazinyl group substituted with a substituted or unsubstituted aryl group.

38. The organic light-emitting device according to claim 23, wherein A¹ and A² are the same groups.

39. The organic light-emitting device according to claim 23, wherein the compound represented by the general formula (1) has a lowest excited triplet energy level (E_{T1}) of 2.90 eV or more.

40. The organic light-emitting device according to claim 23, which is for an organic light-emitting device having a maximum emission wavelength of 360 to 495 nm.

41. The compound according to claim 22, wherein A¹ and A² are independently a heteroaryl group bonding to Ar¹ or Ar² via a carbon atom and containing a triazine ring.

42. The compound according to claim 22, wherein A¹ and A² are independently a triazinyl group substituted with a substituted or unsubstituted aryl group.
